(19) United States
(12) Patent Application Publication
(10) Pub. No.: US 2014/0329252 A1
(43) Pub. Date: Nov. 6, 2014

(54) METHODS AND KITS FOR DIAGNOSING AND/OR PROGNOSING OSTEOARTHRITIS

(71) Applicant: HOPITAL SAINTE-JUSTINE, MONTREAL (CA)

(72) Inventor: Alain Moreau, Montreal (CA)

(21) Appl. No.: 14/351,398

(22) PCT Filed: Oct. 15, 2012
(86) PCT No.: PCT/CA2012/050723

§ 371 Date: Apr. 11, 2014

Related U.S. Application Data
(60) Provisional application No. 61/547,275, filed on Oct. 14, 2011.

(51) Int. Cl. G01N 33/573; G01N 33/569

(52) U.S. Cl. CPC G01N 33/573; G01N 33/56966; G01N 2333/9015; G01N 2333/47; G01N 2800/105
USPC 435/7.4; 435/7.1

(57) ABSTRACT

A method of determining whether a subject is at risk of developing osteoarthritis (OA), said method comprising: determining the cellular localization of a Prohibitin-1 (PHB1) polypeptide and/or Small Ubiquitin-like Modifier (SUMO) polypeptide and/or UBC9, in a cell sample from said subject; and determining whether said subject is at risk of developing OA based on the cellular localization of a PHB1 polypeptide and/or SUMO and/or UBC9 polypeptide, is described.

### CROSS REFERENCE TO RELATED APPLICATIONS

This application is PCT application No. PCT/CA2012/00______ filed on Oct. 15, 2012 and published in English under PCT Article 21(2), which claims benefit of U.S. provisional application Ser. No. 61/547,275, filed on Oct. 14, 2011. All documents above are incorporated herein in their entirety by reference.

### FIELD OF THE INVENTION

The present invention relates to the degradation of joints, and more particularly to the prognosis and/or diagnosis of osteoarthritis (OA).

### REFERENCE TO SEQUENCE LISTING

Pursuant to 37 C.F.R. 1.821(c), a sequence listing is submitted herewith as an ASCII compliant text file named 13200.18_ST25.txt, created on Oct. ______, 2012 and having a size of ______ kilobytes. The content of the aforementioned file is hereby incorporated by reference in its entirety.

### BACKGROUND OF THE INVENTION

The etiology of OA, the most common form of arthritis, remains unclear notwithstanding the multiplicity of factors that have been considered in primary OA (1, 2). At present, it has become increasingly evident that the majority of OA genetic susceptibility loci cannot be attributed only to structural genes or genes regulating bone mass (3-5). These studies have also highlighted the great heterogeneity and differences in the degree of OA heritability between different joint sites (e.g., hand versus knee) and gender. This is also reflected by the multiplicity of loci identified in OA linkage studies and their discrepancies. Moreover, the functional importance of these susceptibility loci has yet to be confirmed and illustrates our incomplete knowledge of the biology of OA.

Diagnosis of OA is generally made based on history and clinical examination to observe signs and symptoms associated with OA such as joint swelling, joint tenderness, decreased range of motion in joints, visible joint damage (i.e., bony growths), etc. X-rays are typically used to confirm the diagnosis of osteoarthritis. The typical changes seen on X-ray include: joint space narrowing, subchondral sclerosis (increased bone formation around the joint), subchondral cyst formation, and osteophytes.

There is a need for novel methods and kits for the assessment of the risk of development, progression and/or severity of OA.

The present description refers to a number of documents, the content of which is herein incorporated by reference in their entirety.

### SUMMARY OF THE INVENTION

In accordance with an aspect of the present invention, there is provided a method of determining whether a subject (e.g., asymptomatic or diagnosed) is at risk of developing (e.g., first symptoms or more severe symptoms) osteoarthritis, said method comprising: determining the cellular localization of a Prohibitin-1 (PHB1) polypeptide and/or Small Ubiquitin-like Modifier (SUMO) polypeptide, and/or increased expression or activity of Ubc9 polypeptide in a blood cell sample from said subject; and determining whether said subject is at risk of developing osteoarthritis based on the cellular localization of a PHB1 polypeptide and/or SUMO polypeptide. In that context, OA patients exhibiting stronger nuclear accumulation of PHB1 and/or SUMO-1, and/or SUMO2 and/or SUMO3 sumoylated proteins and/or Ubc9 expression or activity present a greater risk of disease aggravation (disease staging).

In accordance with another aspect of the present invention, there is provided a method of determining whether a subject is at risk of developing osteoarthritis (OA), said method comprising: determining the cellular localization of a Prohibitin-1 (PHB1) polypeptide and/or Small Ubiquitin-like Modifier (SUMO) polypeptide, in a cell sample from said subject; and determining whether said subject is at risk of developing OA based on the cellular localization of a PHB1 polypeptide and/or SUMO polypeptide.

In an embodiment, the above-mentioned method further comprises determining whether the PHB1 polypeptide and/or SUMO polypeptide nuclear concentration is higher in the subject blood cell sample relative to that in a control blood cell sample; wherein a higher PHB1 polypeptide and/or SUMO polypeptide nuclear concentration in the subject cell sample is indicative that the subject is at risk of developing osteoarthritis.

In a specific embodiment, said method further comprises determining whether the PHB1 polypeptide and/or SUMO polypeptide nuclear concentration is higher in the subject blood cell sample relative to that in a control blood cell sample; wherein a higher PHB1 polypeptide and/or SUMO polypeptide nuclear concentration in the subject cell sample is indicative that the subject is at risk of developing OA.

In a specific embodiment, said method further comprises determining the cellular localization of a promyelocytic leukemia (PML) polypeptide, in the cell sample from said subject, wherein a higher level of co-localization of a SUMO-1 and/or SUMO-2 and/or SUMO-3 polypeptide and the PML polypeptide in nuclear bodies of the cell from said subject is indicative that the subject is at risk of developing OA.

In another specific embodiment, said cell sample (e.g., blood cell sample) is a peripheral blood mononuclear cell (PBMC) sample. In another specific embodiment, said SUMO polypeptide is a SUMO-1 polypeptide.

In another specific embodiment, said SUMO polypeptide is a SUMO-2 polypeptide.

In another specific embodiment, said SUMO polypeptide is a SUMO-3 polypeptide.

In another specific embodiment, a higher level of the SUMO polypeptide in nuclear bodies of the cell from said subject is indicative that the subject is at risk of developing OA.

In another specific embodiment, said method comprises: determining whether the level of co-localization of the SUMO-1 polypeptide and the PHB1 polypeptide in the nuclear bodies is higher relative to that in a control cell; wherein a higher level of co-localization of a SUMO-1 polypeptide and a PHB1 polypeptide in nuclear bodies of the cell from said subject is indicative that the subject is at risk of developing OA.

In accordance with another aspect of the present invention, there is provided a method of determining whether a subject is at risk of developing osteoarthritis (OA), said method comprising: determining the level of an enzyme involved in the sumoylation of protein in a cell sample from said subject; and determining whether said subject is at risk of developing OA based on the level of said enzyme in said cell sample.

In a specific embodiment, said method further comprises determining whether the level of said enzyme is higher in the subject sample relative to that in a control cell sample, wherein a higher level of said enzyme in the subject cell sample is indicative that the subject is at risk of developing OA.

In another specific embodiment, said enzyme is ubiquitin-like protein sumo conjugating enzyme (UBC9).

In accordance with another aspect of the present invention, there is provided a method of determining whether a subject is at risk of developing osteoarthritis, said method comprising: determining whether the level of a SUMO polypeptide in nuclear bodies of a cell from said subject is higher relative to that in a control cell; wherein a higher level of a SUMO polypeptide in nuclear bodies of the cell from said subject is indicative that the subject is at risk of developing osteoarthritis.

In accordance with another aspect of the present invention, there is provided a method of determining whether a subject is at risk of developing osteoarthritis, said method comprising: determining whether the level of co-localization of a SUMO-1 polypeptide and a PHB1 polypeptide in nuclear bodies of a cell from said subject is higher relative to that in a control cell; wherein a higher level of co-localization of a SUMO-1 polypeptide and a PHB1 polypeptide in nuclear bodies of the cell from said subject is indicative that the subject is at risk of developing osteoarthritis.

In accordance with another aspect of the present invention, there is provided a method of determining whether a subject is at risk of developing osteoarthritis (OA), said method comprising: determining whether the level of co-localization of a SUMO-1 and/or SUMO-2 and/or SUMO-3 polypeptide and a PML polypeptide in nuclear bodies of a cell from said subject is higher relative to that in a control cell; wherein a higher level of co-localization of a SUMO-1 and/or SUMO-2 and/or SUMO-3 polypeptide and a PML polypeptide in nuclear bodies of the cell from said subject is indicative that the subject is at risk of developing OA.

In accordance with another aspect of the present invention, there is provided a method of determining whether a subject is at risk of developing osteoarthritis, said method comprising: determining whether (i) the amount of PML nuclear bodies in a cell from said subject is higher relative to that in a control cell and/or (ii) the size of PML nuclear bodies in a cell from said subject is larger relative to that in a control cell; wherein a higher amount and/or larger size of PML nuclear bodies in the cell from said subject is indicative that the subject is at risk of developing osteoarthritis.

In accordance with another aspect of the present invention, there is provided a method of determining whether a subject is at risk of developing osteoarthritis, said method comprising: determining whether the level of an enzyme involved in sumoylation (e.g., ubiquitin-like protein SUMO conjugating enzyme (UBC9)) in a cell sample from said subject; and determining whether said subject is at risk of developing osteoarthritis based on the level of said enzyme involved in sumoylation in said cell sample. In an embodiment, the above-mentioned method further comprises determining whether the level of said enzyme is higher in the subject cell sample relative to that in a control cell sample; wherein a higher level of said enzyme in the subject cell sample is indicative that the subject is at risk of developing osteoarthritis.

In an embodiment, the above-mentioned method further comprises determining whether the level of said enzyme is higher in the subject sample relative to that in a control cell sample; wherein a higher level of said enzyme in the subject cell sample is indicative that the subject is at risk of developing OA. In another embodiment, said enzyme is ubiquitin-like protein SUMO conjugating enzyme (UBC9).

In another embodiment, of the above-mentioned methods, said cell is an articular chondrocyte, a growth plate chondrocyte, an osteoblast, a skeletal myoblast, synoviocyte or a blood cell.

In another embodiment, of the above-mentioned methods, said cell sample is an articular chondrocyte sample, a growth plate chondrocyte sample, an osteoblast sample, a skeletal myoblast sample, a synoviocyte sample or a blood cell sample.

In another embodiment, said cell or cell sample is a peripheral blood mononuclear cell (PBMC) sample.

In another embodiment, said cell or cell sample is a leucocytes sample.

In accordance with another aspect of the present invention, there is provided a method of determining whether a subject is at risk of developing osteoarthritis, said method comprising: determining the level of PHB1 in a blood sample from said subject; and determining whether said subject is at risk of developing osteoarthritis based on the level of PHB1 in said blood sample; wherein a lower level of PHB1 in the subject blood sample is indicative that the subject is at risk of developing osteoarthritis.

In an embodiment, the above-mentioned methods further comprise identifying a subject suspected of having osteoarthritis (OA).

In an embodiment, the above-mentioned methods further comprise identifying a subject suspected of having primary osteoarthritis (OA).

In an embodiment of the above-mentioned methods, the OA is knee joint arthritis, hip joint arthritis or temporo-mandibular joint arthritis. In an embodiment of the above-mentioned methods, the OA is knee joint arthritis. In an embodiment of the above-mentioned methods, the OA is hip joint arthritis. In an embodiment of the above-mentioned methods, the OA is primary OA.

The method of any one of claims 1 to 20, wherein the determining of whether the subject is at risk of developing OA determines whether the subject is at risk of developing a more severe primary OA symptoms at a future time.

In accordance with another aspect of the present invention, there is provided a method of selecting a compound, said method comprising (a) contacting a test compound with a blood cell sample; and (b) determining a PHB1 polypeptide and/or SUMO polypeptide nuclear localization in the blood cell; wherein the test compound is selected if the PHB1 polypeptide and/or SUMO polypeptide nuclear localization in the cell sample is decreased in the presence of the test compound relative to in the absence thereof.

In accordance with another aspect of the present invention, there is provided a method of selecting a compound, said method comprising (a) contacting a test compound with a cell sample; and (b) determining a level of a SUMO polypeptide in nuclear bodies in the cell; wherein the test compound is selected if the level of SUMO polypeptide in nuclear bodies is decreased in the presence of the test compound relative to in the absence thereof.

In accordance with another aspect of the present invention, there is provided a method of selecting a compound, said method comprising (a) contacting a test compound with a cell sample; and (b) determining a level of co-localization of a SUMO-1 polypeptide and a PHB1 polypeptide in nuclear bodies in the cell; wherein the test compound is selected if the level of co-localization of SUMO-1 polypeptide and PHB1 polypeptide in nuclear bodies is decreased in the presence of the test compound relative to in the absence thereof.

In accordance with another aspect of the present invention, there is provided a method of selecting a compound, said method comprising (a) contacting a test compound with a cell sample; and (b) determining (i) an amount of promyelocytic leukemia (PML) nuclear bodies in the cell and/or (ii) the size of PML nuclear bodies in the cell; wherein the test compound is selected if the amount and/or size of PML nuclear bodies is decreased in the presence of the test compound relative to in the absence thereof.

In accordance with another aspect of the present invention, there is provided a method of selecting a compound, said method comprising (a) contacting a test compound with a cell sample; and (b) determining a level of an enzyme involved in sumoylation (e.g., UBC9) in the cell sample; wherein the test compound is selected if the level of said enzyme in the cell sample is decreased in the presence of the test compound relative to in the absence thereof.

In accordance with another aspect of the present invention, there is provided a method of selecting a compound, said method comprising (a) administering a test compound to a subject; and (b) determining a level of PHB1 in a blood sample from said subject; wherein the test compound is selected if the level of PHB1 in the blood sample is increased in the presence of the test compound relative to in the absence thereof.

In another specific embodiment, the selected test compound is potentially useful in the treatment of primary osteoarthritis.

In a specific embodiment of the methods, the osteoarthritis is knee joint arthritis, hip joint arthritis or temporo-mandibular joint arthritis. In another specific embodiment, the osteoarthritis is knee joint arthritis. In another specific embodiment, the osteoarthritis is hip joint arthritis. In another embodiment, the osteoarthritis is primary osteoarthritis.

In an embodiment, the above-mentioned cell is an articular chondrocyte, a growth plate chondrocyte, an osteoblast, a skeletal myoblast, a synoviocyte or a blood cell. In a further embodiment, the blood cell is a peripheral blood mononuclear cell (PBMC).

In a specific embodiment of the methods, the subject is a woman.

In accordance with another aspect of the present invention, there is provided a kit comprising a ligand specific to a Prohibitin-1 (PHB1) polypeptide and/or Small Ubiquitin-like Modifier (SUMO) polypeptide, and/or UBC9 polypeptide and instructions to use the ligand to predict whether a subject is at risk for developing osteoarthritis.

In a specific embodiment of the kit, the kit comprises at least two of a ligand specific to a Prohibitin-1 (PHB1) polypeptide, a ligand specific to a Small Ubiquitin-like Modifier (SUMO) polypeptide (SUMO 1, 2 and/or 3), and a ligand specific to a UBC9 polypeptide. In a specific embodiment of the kit, the kit comprises a ligand specific to a Prohibitin-1 (PHB1) polypeptide, a ligand specific to a Small Ubiquitin-like Modifier (SUMO) polypeptide, and a ligand specific to a UBC9 polypeptide. In a specific embodiment the ligand is a ligand specific to a Prohibitin-1 (PHB1) polypeptide and/or Small Ubiquitin-like Modifier (SUMO) polypeptide, and/or UBC9 polypeptide.

The articles “a,” an and the are used herein to refer to one or to more than one (i.e., to at least one) of the grammatical object of the article.

The terms “including” and “comprising” are used herein to mean, and re used interchangeably with, the phrases “including but not limited to” and “comprising but not limited to”.

The terms such as are used herein to mean, and is used interchangeably with, the phrase “such as but not limited to”.

As used herein the term “osteoarthritis” refers to a form of arthritis involving the deterioration of the cartilage that cushions the ends of bones within joints. It is also called degenerative arthritis, degenerative joint disease or hypertrophic arthritis. This term includes early onset of osteoarthritis. Worldwide, osteoarthritis is the most common joint disorder. In western countries, radiographic evidence of this disease is present in the majority of persons by 65 years of age and in about 80 percent of persons more than 75 years of age (33). Approximately 11 percent of persons more than 64 years of age have symptomatic osteoarthritis of the knee (34).

As used herein the terms “early onset of osteoarthritis” refer to a form of osteoarthritis that either is first diagnosed at 40 years of age or earlier or that leads to knee joint replacement of the subject before he is 55 years old.

As used herein the terms “risk of developing osteoarthritis” refers to a predisposition of a subject of presenting primary OA symptoms and/or more severe primary OA symptoms at a future time (disease staging). Similarly, the “risk of developing osteoarthritis in a joint where Pitx1 is normally expressed” refers to a risk for a subject of presenting primary OA symptoms, and/or more severe primary OA symptoms at a future time in a joint where Pitx1 is normally expressed.

As used herein the terms “primary OA” when used to qualify knee/hip joint OA refer to knee/hip joint OA due to a disease or degeneration for instance as opposed to secondary knee/hip joint OA resulting from trauma, joint overuse, obesity, etc.

As used herein the term “subject” is meant to refer to any mammal including human, mice, rat, dog, cat, pig, monkey, horse, etc. In a particular embodiment, it refers to a human. In another particular embodiment, it refers to a horse and more specifically a racing horse.

As used herein the terms “predisposition for developing a disease or condition” refers to a predisposition of a subject of presenting symptoms of the disease or condition and/or more severe symptoms of the disease or conditions at a future time.

As used herein the terms “control sample” are meant to refer to a sample that does not come from a subject known to suffer from the disease or disorder or from the subject under scrutiny but before the subject had the disease or disorder. In methods of diagnosing a predisposition of a subject to develop a disease or disorder, the sample may also come from the subject under scrutiny at an earlier stage of the disease or disorder. The term “control sample” may also refer to a pre-determined, control value recognized in the art or established based on levels measured in one or a group of control subjects. The corresponding control level/value may be adjusted or normalized for age, gender, race, or other parameters. The control level can thus be a single number/value, equally applicable to every patient individually, or the control level can vary, according to specific subpopulations of patients.

As used herein the term “cell sample” is meant to refer to a sample containing any type of cell wherein, in a subject affected by OA, PHB1, SUMO (SUMO-1 and/or SUMO-2 and/or SUMO-3) and/or UBC9 pathologically accumulates in the cell nuclei (e.g., in nuclear bodies). Without being so limited, it includes articular chondrocytes, growth plate chondrocytes, osteoblasts, skeletal myoblasts, synoviocytes, blood cells (e.g., PBMCs). As used herein the term “articular chondrocyte” is meant to refer to chondrocytes found in joints.

As used herein, the term “blood cell sample” refers to a sample containing cells normally found in blood, and includes for example peripheral blood mononuclear cells (PBMCs) as well as particular cell types such as lymphocytes (T cells, B cells, NK cells), monocytes, basophils, and dendritic cells, or any mixture thereof. In an embodiment, the above-mentioned blood cell sample may be submitted to one or more cell depletion or enrichment steps, so as to enrich the sample in one or more cell types of interest.

As used herein the term “blood sample” is meant to refer to a sample derived from blood, and include for example whole blood, or to a fraction thereof, such as serum, plasma and the like. It also refers to any sample that may be obtained following one or more purification, enrichment, and/or treatment steps using blood (obtained by venous puncture, for example) as starting material. In an embodiment, the blood sample is a plasma sample.

As used herein the term “not clinically diagnosed with osteoarthritis” is meant to refer to a subject that was never diagnosed with OA using a clinical method such as an imaging method like X-ray, and magnetic resonance imaging (MRI). In particular, for diagnosing hip OA, a current clinical method recommended by the American College of Rheumatology includes hip pain and at least 2 of the following 3 features: ESR<20 mm/hour; radiographic femoral or acetabular osteophytes; and radiographic joint space narrowing (superior, axial, and/or medial). In particular, for diagnosing knee OA, there are three methods currently recommended by the American College of Rheumatology Clinical and laboratory method: knee pain and at least 5 of the following 9 features: age >50 years, stiffness <30 minutes, crepitus, bony tenderness, bony enlargement, no palpable warmth, ESR <40 mm/hour, RF <1:40; and SF OA; 2) Clinical and radiographic: knee pain, and at least 2 of the following 3 features, Age >50 years; stiffness <30 minutes; crepitus; +osteophytes; and 3) Clinical: knee pain and at least 3 of the following 6 features: age >50 years, stiffness <30 minutes, crepitus, bony tenderness, bony enlargement, no palpable warmth.

As used herein the terminology “purified”, “isolated”, “purification” or “isolation” in the expressions “purified polypeptide”, “isolated polypeptide”, “isolated protein”, “purified complexes”, “isolated complexes” or “tandem affinity purification” means altered “by the hand of man” from its natural state (i.e. if it occurs in nature, it has been changed or removed from its original environment) or it has been synthesized in a non-natural environment (e.g., artificially synthesized). These terms do not require absolute purity (such as a homogeneous preparation) but instead represents an indication that it is relatively more pure than in the natural environment. For example, a protein/peptide naturally present in a living organism is not “purified” or “isolated”, but the same protein separated (about 90-95% pure at least) from the coexisting materials of its natural state is “purified” or “isolated” as this term is employed herein.

Sumoylation is a post-translational modification in which a molecule called SUMO (Small Ubiquitin-like MOdifier) is covalently but reversibly linked to a lysine residue in a process similar to ubiquitination. SUMO proteins are ubiquitous in eukaryotes and highly conserved from yeast to humans. Generally, sumoylation seems to have an inhibitory effect on gene transcription and it was proposed that sumoylation could act on various transcription factors to promote their interaction with co-repressors (Gill G. Curr. Opin. Genet. Dev. 2005; 15:536-541). In vertebrates, there are four isoforms of SUMO proteins named SUMO-1 to SUMO-4 (Gill 2005, supra, FIGS. 19 to 22). SUMOs are attached to their target proteins in a three-step process implying an activation enzyme E1, a conjugation enzyme E2 and an E3 ligase. In humans, E1 is composed of two subunits (SAE1/SEA2), the unique conjugation E2 enzyme is called UBC9 and there are at least five known E3 ligases: PIAS1 (protein inhibitor of activated signal transducer), which is the prototype of a family that encompasses three additional members (PIAS3, PIASy and PIASx), Pc2 (human Polycomb member 2) and Ran-BP2. There also exist at least seven SUMO-specific proteases in humans named SENP-1 to SENP-8.

In an embodiment, the above-mentioned SUMO polypeptide is a SUMO-1, SUMO-2, SUMO-3 and/or SUMO-4 polypeptide. In an embodiment, the above-mentioned SUMO polypeptide is a SUMO-1, SUMO-2, and/or SUMO-3 polypeptide. In a further embodiment, above-mentioned SUMO polypeptide is a SUMO-1 polypeptide.

In an embodiment, the above-mentioned enzyme involved in sumoylation is an activation enzyme E1, a conjugation enzyme E2 and/or an E3 ligase. In a further embodiment, the above-mentioned enzyme involved in sumoylation is a conjugation enzyme E2, in a further embodiment UBC9.

### Diagnostic or Prognostic Methods

A method for diagnosing or screening for the presence of a disease or disorder or a predisposition for developing the disease or disorder in a subject (“risk of developing”), which disease or disorder is characterized by an aberrant amount, activity, protein composition, intracellular localization and/or formation of a complex, comprising the steps of: (1) comparing the amount of, activity of, protein composition of, intracellular localization (e.g., in nuclear bodies such as PML nuclear bodies) of, and/or formation of said complex (e.g., SUMO-1 and/or -2 and/or -3 with at least another protein (e.g., PML, PHB1)) in a sample from the subject with that in a control sample, wherein a difference in said amount, activity, protein composition of, intracellular localization and/or formation of said complex as compared to that in the control sample is indicative that the subject has the disease or disorder or a predisposition for developing the disease or condition. A comparison of amount, activity, protein composition, intracellular localization and/or formation of a complex of certain proteins between various OA patients may also provide means of classifying/stratifying the patients. Hence for example, when comparing two OA subjects, detecting a higher level of PHB1 and/or SUMO-1 and/or SUMO-2 and/or SUMO-3 and/or UBC9 in the first OA subject than in the second OA subject is an indication that the first OA subject has a higher risk of developing OA or a risk of developing a more severe OA form than the second OA subject.

In a specific embodiment, the control sample is selected from a sample from the subject at an earlier stage of the disease or disorder or before the subject had the disease. In another embodiment, the control sample is from a different subject that does not have the disease or disorder or predisposition to develop the disease or condition.

The amount and/or localization of PHB1, SUMO (e.g., SUMO-1) and/or UBC9 may be determined using any known method in the art. In an embodiment, the amount and/or localization of PHB1, SUMO (e.g., SUMO-1) and/or UBC9 is determined at the protein/polypeptide level, for example using a molecule capable of specifically binding to a PHB1, SUMO (e.g., SUMO-1) or UBC9 polypeptide. PHB1, SUMO (e.g., SUMO-1) or UBC9 polypeptide expression levels may be determined using any standard methods known in the art. Non-limiting examples of such methods include Western blot, tissue microarray, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, immunofluorescence, matrix-assisted laser desorption/ionization time-of-flight (MALDI-TO F) mass spectrometry, microcytometry, microscopy, fluorescence-activated cell sorting (FACS), flow cytometry, and assays based on a property of the protein including but not limited to DNA binding, ligand binding, or interaction with other protein partners.

In an embodiment, the molecule capable of specifically binding to a PHB1, SUMO (e.g., SUMO-1) or UBC9 polypeptide is an antibody specifically binding to, or specifically recognizing, a PHB1, SUMO (e.g., SUMO-1) or UBC9 polypeptide.

As used herein, the term “antibody” refers to an antibody that specifically binds to (interacts with) a protein of interest (PHB1, SUMO (1, 2 and/or 3) and/or UBC9) and displays no substantial binding to other naturally occurring proteins other than the ones sharing the same antigenic determinants. The term antibody or immunoglobulin is used in the broadest sense, and covers monoclonal antibodies (including full length monoclonal antibodies), polyclonal antibodies, multispecific antibodies, and antibody fragments so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (VH, VH-VH), anticalins, PepBodies™, antibody-T-cell epitope fusions (Troybodies) or Peptibodies. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories).

The present invention also relates to methods for the determination of the level of expression of transcripts or translation product of a gene such as SUMO, PHB1 or UBC9. The present invention therefore encompasses any known method for such determination including real time PCR and competitive PCR, in situ PCR, SAGE, Northern blots, in situ hybridization, Southern blot, nuclease protection, plaque hybridization and slot blots.

The present invention also concerns isolated nucleic acid molecules including probes. In specific embodiments, the isolated nucleic acid molecules have no more than 300, or no more than 200, or no more than 100, or no more than 90, or no more than 80, or no more than 70, or no more than 60, or no more than 50, or no more than 40 or no more than 30 nucleotides. In specific embodiments, the isolated nucleic acid molecules have at least 20, or at least 30, or at least 40 nucleotides. In other specific embodiments, the isolated nucleic acid molecules have at least 20 and no more than 300 nucleotides. In other specific embodiments, the isolated nucleic acid molecules have at least 20 and no more than 200 nucleotides. In other specific embodiments, the isolated nucleic acid molecules have at least 20 and no more than 100 nucleotides. In other specific embodiments, the isolated nucleic acid molecules have at least 20 and no more than 90 nucleotides. In other specific embodiments, the isolated nucleic acid molecules have at least 20 and no more than 80 nucleotides. In other specific embodiments, the isolated nucleic acid molecules have at least 20 and no more than 70 nucleotides. In other specific embodiments, the isolated nucleic acid molecules have at least 20 and no more than 60 nucleotides. In other specific embodiments, the isolated nucleic acid molecules have at least 20 and no more than 50 nucleotides. In other specific embodiments, the isolated nucleic acid molecules have at least 20 and no more than 40 nucleotides. In other specific embodiments, the isolated nucleic acid molecules have at least 20 and no more than 30 nucleotides. In other specific embodiments, the isolated nucleic acid molecules have at least 30 and no more than 300 nucleotides. In other specific embodiments, the isolated nucleic acid molecules have at least 30 and no more than 200 nucleotides. In other specific embodiments, the isolated nucleic acid molecules have at least 30 and no more than 100 nucleotides. In other specific embodiments, the isolated nucleic acid molecules have at least 30 and no more than 90 nucleotides. In other specific embodiments, the isolated nucleic acid molecules have at least 30 and no more than 80 nucleotides. In other specific embodiments, the isolated nucleic acid molecules have at least 30 and no more than 70 nucleotides. In other specific embodiments, the isolated nucleic acid molecules have at least 30 and no more than 60 nucleotides. In other specific embodiments, the isolated nucleic acid molecules have at least 30 and no more than 50 nucleotides. In other specific embodiments, the isolated nucleic acid molecules have at least 30 and no more than 40 nucleotides.

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally known (62,63). Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

As used herein the terms “detectably labeled” refer to a marking of a probe in accordance with the presence invention that will allow the detection of the mutation of the present invention. Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (64). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

The present invention also relates to methods of selecting compounds. As used herein the term “compound” is meant to encompass natural, synthetic or semi-synthetic compounds, including without being so limited chemicals, macromolecules, cell or tissue extracts (from plants or animals), nucleic acid molecules, peptides, antibodies and proteins.

The present invention also relates to arrays. As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

As used herein “array of nucleic acid molecules” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Any known nucleic acid arrays can be used in accordance with the present invention. For instance, such arrays include those based on short or longer oligonucleotide probes as well as cDNAs or polymerase chain reaction (PCR) products (52). Other methods include serial analysis of gene expression (SAGE), differential display, (53) as well as subtractive hybridization methods (54), differential screening (DS), RNA arbitrarily primer (RAP)-PCR, restriction endonucleolytic analysis of differentially expressed sequences (READS), amplified restriction fragment-length polymorphisms (AFLP).

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see 64 for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Washing with a solution containing tetramethylammonium chloride (TeMAC) could allow the detection of a single mismatch using oligonucleotide hybridization since such mismatch could generate a 10° C. difference in the annealing temperature. The formulation to determine the washing temperature is Tm (° C.)=]−682 (L⁻¹)+97 where L represents the length of the oligonucleotide that will be used for the hybridization.

The present invention also encompasses arrays to detect and/or quantify the nuclear localization of proteins including PHB1, SUMO and UBC9. Such arrays include protein micro- or macroarrays, gel technologies including high-resolution 2D-gel methodologies, possibly coupled with mass spectrometry (55), imaging system at the cellular level such as microscopy combined with a fluorescent labeling system.

The present invention also includes the use of tissue biopsy to determine the nuclear accumulation of PHB1, SUMO and UBC9 within articular chondrocytes, growth plate chondrocytes, osteoblasts, skeletal myoblasts and synoviocytes. For instance, cartilage biopsy could be performed during arthroscopy procedure to assess OA or its progression by immunofluorescence microscopy to determine the nuclear localization of PHB1, SUMO and UBC9. This method could be useful for instance when arthroscopy procedure is required to establish a clinical diagnostic. Alternatively, a muscle biopsy in lower limbs could be used to test whether or not PHB1, SUMO and UBC9 are accumulated in the nuclei of myoblasts. This method would advantageously be less invasive than a regular arthroscopy. The determination of the cellular localization or concentration of a protein as disclosed herein (e.g., PHB1, SUMO and/or UBC9) is typically performed either by a) preparing a nuclear extract of a subject sample and determining concentration of PHB1, SUMO and UBC9; or by (b) determining the localization of PHB1, SUMO and UBC9 by immunohistochemistry. Cellular localization or concentration of these molecules may also be detected by other imaging or detection methods enabling the visualization and quantification of biomolecules, such as flow cytometry.

The present invention relates to a kit for diagnosing OA and/or predicting whether a subject is at risk of developing OA comprising an isolated nucleic acid, a protein or a ligand such as an antibody in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the subject sample (DNA genomic nucleic acid, cell sample or blood samples), a container which contains in some kits of the present invention, the probes used in the methods of the present invention, containers which contain enzymes, containers which contain wash reagents, and containers which contain the reagents used to detect the extension products. The present invention also relates to a kit comprising the antibodies which are specific to PHB1, SUMO and/or UBC9. Kits of the present invention may also contain instructions to use these probes and or antibodies to diagnose OA or predict whether a subject is at risk of developing OA.

As used herein, the term “ligand” broadly refers to natural, synthetic or semi-synthetic molecules. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The ligand appropriate for the present invention can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “ligand”. For example, peptidomimetics, well known in the pharmaceutical industry and generally referred to as peptide analogs can be generated by modeling as mentioned above.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1 shows mean levels of PHB1 in plasma samples obtained from knee (n=43) and hip (n=44) osteoarthritis patients and age-matched healthy subjects (n=31). Values were generated with in-house kit;

FIG. 2 shows data analysis of plasmatic PHB1 levels (PHB). Descriptive values of PHB1 plasma levels are shown by sex and health group expressed in ng/ml;

FIG. 3 shows Statistical analysis system (SAS). Output statistical analysis using data for women only as an interaction exist between levels of PHB1 and sex. In the logistic model used, the outcome is OA or Healthy status and the predictor is PHB1 alone or in combination with co-variate(s);

FIG. 4 shows the distribution of PHB1 in the cytosol (C-X) and nucleus (N-X) of human lymphocytes from two OA patients and a control subject. Proteins were resolved by SDS-PAGE and PHB1 protein was detected using an anti-PHB1 antibody. GAPDH was used as a cytoplasmic marker. The demographic and clinical data corresponding to each patient tested are indicated in the table. The Kellgren-Lawrence (KL) score is a radiographic score used to differentiate the severity of OA (from 1 to 4, wherein 4 corresponds to the most severe form of OA; N/A=data not available). Early stage of OA is defined as patients exhibiting a KL score 2, while late stage OA patients is defined as those exhibiting a KL score ≧3;

FIG. 5 shows the accumulation of PHB1 in clusters in lymphocyte nucleus. (A) PHB1 (green) (arrows point to examples of PHB-1 agglomerates/clusters (i.e. PHB1 positive nuclear bodies)) was visualized by immunofluorescence using confocal microscopy and pictures were taken at an optical section localising to the center of the nuclear region. Upper panels represent representative examples of agglomerates in lymphocytes derived from a healthy control (left) and a subject having osteoarthritis (OA, right) affected patient. Lower panels correspond to higher magnification of lymphocytes derived from a healthy donor (left) or an OA patient (right) immunostained for PHB1. Arrows indicate nuclear aggregate/clusters of PHB1 seen in these cells. (B) Quantification of the number of PHB1 agglomerates (paler) per lymphocyte nucleus (darker) per patient (OA, n=3; Healthy, n=4). Represented is the average of number of aggregate/clusters per cell per patient with its associated standard error. Three to thirty cells were analysed per patient and three or four patients were analysed per group (*P<0.01). (C) Frequency distribution for the cells analysed in (B). The Y-axis represents the number of cells with the given number of aggregate/clusters (X-axis). Overlay is a polynomial regression curve for each patient group. Images were obtained with a Zeiss™ microscope (LSM 510 META) and its associated LSM acquisition software (Release 2.5) using a 63× objective. Images were exported as Tiff files for quantitative analysis. The number of PHB1 nuclear aggregates, regardless of their size, was quantified manually per cell per patient. These data were reported either as frequency distribution i.e. as numbers of cells that have a given number of nuclear aggregates/clusters per patient group (C) or as the average means number of aggregates/clusters per cell in patient groups (B) with its associated standard error. Based on the frequency distribution, the Poisson frequency distribution per patient group was calculated using Microsoft Excel™ spreadsheet (Microsoft Office 2007). To facilitate the visual comparison between the curves, these curves were calculated for 100 cells. Comparison among patient groups for the average number of PHB1 nuclear aggregates was performed using the procedure GLM of the SAS software v8.02 (Cary, N.C.);

FIG. 6 shows that the signal intensity for PHB1, which denotes protein levels, is increased in whole leucocytes of OA patients (lymphocytes and monocytes). (A): shows the sensitivity value of the microscope camera (gain detector) for each sample analysed. All samples were obtained from women. The lower the sensitivity value, the brighter the signal. For healthy subject (n=9) and for OA patients (n=7). Horizontal bars represent average value in each group (Healthy subjects=1015; OA subjects=947). Analysis of the gain detector values was performed using logistic regression with the software SAS v9.2. (B): shows a Table summarizing the covariate parameters. (C): shows the analysis of the gain detector values was performed using logistic regression with the software SAS v9.2. Represented is the p value for the gain detector variable adjusted for the indicated co-variable;

FIG. 7 shows that the proportion of leucocytes nuclei expressing low levels of PHB1 is decreased in OA patients (n=5) vs. controls (healthy subjects) (n=3);

FIG. 8 shows a comparison between the number of nuclear PHB1 agglomerates in leucocytes of control subjects (n=5) and OA patients (n=6);

FIG. 9 shows that PHB1 mostly accumulates in nucleus of OA chondrocytes. (A) PHB1 immunostaining performed on cartilage sections from control (top panels) or OA (bottom panels) subjects. Arrows show cells with a positive nuclear PHB1 signal. Cartilage sections were counterstained with Harris Modified Hematoxylin. (B) Quantification of the percentage of cells from control subjects (n=3) and OA subjects (n=5) showing PHB1 nuclear signal. Asterisks represent a significant increase in PHB1 nuclear signal (Mann-Whitney U test: * p<0.05). (C) Cytoplasmic (C) and nuclear (N) protein extracts of primary chondrocytes from one control subject (n=1) and three different OA patients (n=3). Proteins were resolved by SDS-PAGE and PHB1 protein was detected using anti-PHB1 antibody. GAPDH, F1-ATPase and Lamin A/C were used as cytoplasmic, mitochondrial and nuclear marker respectively. (D) Immunofluorescence against PHB1 and TOM20, a mitochondrial marker, on primary chondrocytes from one control subject (n=1) and OA subjects (n=2). PHB1 signal appears in green and TOM20 signal in red. DAPI was used as a DNA marker. (E) PHB1 signal quantification of immunofluorescence results. Data is presented as the percentage of the signal which co-localizes with DAPI signal when compared with the total PHB1 signal. (F) Real time RT-PCR against PHB1 gene in chondrocytes from four healthy subjects (n=4) and nineteen OA patients (n=19) showing that PHB1 gene expression (RNA) is not increased in OA subjects. Black marks represent the median value for each group; (G) Western blot for total levels of PHB1 in leucocyte. Alpha-tubulin is used as loading control;

FIG. 10 shows the distribution of PHB1 in Cytosol (C-X) and Nucleus (N-X) of human articular chondrocytes from OA patients (n=6) and a control subject (n=1). Proteins were resolved by SDS-PAGE and PHB1 protein was detected using anti-PHB1 antibody. GAPDH and Lamin A/C were used as cytoplasmic and nuclear marker respectively. Demographic and clinical data corresponding to each patient tested are indicated below. The Kellgren-Lawrence (KL score) is a radiographic score used to differentiate the severity of OA (1 to 4), where 4 score corresponds to the most severe OA form; N/A data not available); Early stage of OA is defined as patients exhibiting a KL score ≦2, while late stage OA patients is defined as those exhibiting a KL score ≧3;

FIG. 11 shows the increase in nuclear accumulation of PHB1 in knee joint articular chondrocytes of aging STR-ORT mice (n=?), a mouse model of osteoarthritis. Osteoarthritis symptoms in STR-ORT mice are known to occur at about week 30. (A) Microphotographs of STR-ORT mice knee sections stained with Safranin O to visualize the proteoglycan content and the overall knee cartilage structure (M: meniscus; SB; subchondral bone). Mice were aged from 8 to 16 weeks. (B) Representative immunohistochemistry pictures for PHB1 (darker) on paraffin embedded knee sections of STR-ORT mice. Large arrows show cells with nuclear accumulation of PHB1. Small arrows show PHB1 positive cells with no accumulation in the nucleus. Dash line represents the border between cartilage and the subchondral bone (SB). (C) Graphical representation of the results depicted in B (STR-ORT n=7 and Control n=1). (D) qPCR analysis of Pitx1 expression in knees sections of STR-ORT mice (n=5) and control (n=5);

FIG. 12 shows Pitx1 gene repression by PHB1 in C28/I2 chondrocytes cell line. PITX1 mRNA (A) or proteins (B) level from C28/I2 cells stably overexpressing Flag-PHB1 or vector alone. In A, real time RT-PCR was performed against Pitx1 gene. Data is presented as PITX1 mRNA relative quantification and error bars represents standard deviation of triplicates (paired t test: *p<0.01; ** p<0.05). In B, immunoblots of FLAG epitope and PITX1. β-tubuline protein was used as endogenous control. (C-D) Luciferase assays in C28/I2 cell line transiently transfected with PITX1 (−3895/+61 bp)-promoter-luciferase reporters or with luciferase plasmid containing smaller fragments (in C, fragment −3034/+61, −1577/+61 bp, −729/+61 bp, −524/+61, −374/+61 by or −84/+61 by and in D, fragment −729/+61). In C, cells were co-transfected with Flag-PHB1 expressing vector or an empty control vector. In D, cells were co-transfected with Flag-PHB1 expressing vector (Vector/PHB) or an empty control vector (Vector/Vector) and with pBabe plasmid expressing ER fused to E2F1 (E2F1/PHB) or the empty control vector (E2F1/Vector), and induced with 4-hydroxytamoxifen (OHT) for 24 h. Data represents mean and standard deviation of three independent experiments. Asterisks represent a significant decrease in luciferase activity (paired t test: * p<0.05; ** p<0.01) compared to control cells. (E) ChIP assay showing the preferential co-localization of PHB1 on the distal promoter elements of the human PITX1 gene. Real-time PCR analysis was performed after chromatin immunoprecipitation assay against PHB1. Different primers were used to amplify specific PITX1 promoter regions. Data is presented as DNA relative quantification compared to the mean amount of DNA present after immunoprecipitation of control cells.

FIG. 13 shows the rescue of Pitx1 expression in OA articular chondrocytes through PHB1 inhibition. Real time RT-PCR performed against PITX1 gene in chondrocytes from four different OA patients (n=4) transfected with control siRNA or PHB1 siRNA. Data represents mean and standard deviation of three independent experiments. RQ=Relative Quantity. Asterisks represent a significant increase between control and siPHB1 transfected cells (paired t test: *p<0.02);

FIG. 14 shows that specific SUMO proteins accumulate in nuclei of OA articular chondrocytes. (A) Immunoblot of PHB-1 and three SUMOs (PanSumos) were performed using nuclear extract (N) and cytoplasmic extract (C) of articular chondrocytes from a healthy subject (traumatic case non-arthrosic) (n=1), a RA patient (n=1) and two OA patients (n=2). (B) Immunofluorescence staining against SUMO-1 and SUMO-2/3 (the same antibody detect both SUMO-2 and 3) carried out on articular chondrocytes of OA patients and control subjects. Representative staining are shown. OA chondrocytes show a strong nuclear accumulation of SUMO-1 and SUMO-2/3 proteins in the nuclear bodies;

FIG. 15 shows that the SUMO-1 protein strongly accumulates in the nuclei of articular chondrocytes of OA patients compared to healthy subjects. (A) Immunoblot of PHB1 and SUMO-1 were performed using nuclear (N) and cytoplasmic (C) extracts from a healthy patient (n=1) and three OA patients (n=3). Chondrocytes of OA patients show nuclear accumulation of PHB1 and an increase in total sumoylation in the nuclear fraction. (B) Immunofluorescences (IF) against PHB1 and SUMO-1 carried out on the articular chondrocytes of patient OA11. Upper panel show healthy chondrocytes and lower panels show affected chondrocytes;

FIG. 16 shows that PHB1 co-localizes with SUMO-1 in nuclear bodies of OA articular chondrocytes. Double Immunofluorescence stainings against PHB1 and SUMO-1 (A) and PHB1 and SUMO-2/3 (B) were performed on articular chondrocytes of OA patients and control subjects. In OA patients the SUMOs proteins accumulate in nuclear bodies, while control subjects show little or no accumulation of SUMOs in nuclear bodies. PHB1 is co-localized with SUMO-1 while no co-localization was found with SUMO-2/3 in the nuclei of OA chondrocytes (panel B);

FIG. 17 shows that SUMO proteins accumulate in PML nuclear bodies in OA articular chondrocytes. Double immunofluorescence stainings against PML nuclear bodies (PML positive nuclear bodies) and SUMO-1 (A), and PML nuclear bodies and SUMO-2/3 (B) were performed on articular chondrocytes of patients OA and control. In OA chondrocytes, nuclear accumulation of SUMO-2/3 proteins is localized almost solely in PML nuclear bodies while the accumulation of SUMO-1 is in all nucleus including PML nuclear bodies. In OA chondrocytes, PML nuclear bodies are different in size and sometimes they adopt a ring structure, as indicated by an arrow (Panel A). SUMO-1 and SUMO-2/3 both are co-localized in PML in these structures but only in OA chondrocytes;

FIG. 18 shows that PML and PHB1 in human articular chondrocytes from OA patients do not co-localize in nuclei. Double fluorescence staining of OA and control human articular chondrocytes with antibodies against PML and PHB1 shows that PHB1 is accumulated mostly in nuclei of OA chondrocytes like PML although it does not co-localize with PML nuclear bodies;

FIG. 19 shows SUMO-1 expression in leucocytes from 22 weeks old C57BI/6 (n=1) (a mouse not presenting OA symptoms) or STR-ort male mice (n=1) (a mouse model of osteoarthritis). Leucocytes were isolated from blood samples and immunostained for SUMO-1. Nuclei were stained with Draq5. Leucocytes (lymphocytes and monocytes) were isolated by Ficoll gradient, centrifuged (300 g during 6 minutes) on 8-well slides coated>>with poly-D-lysine. Cells were washed twice in PBS and fixed in 4% Paraformaldehyde at Room temperature for 7 minutes, permeabilised in 0.3% Triton X for 5 minutes at room temperature, wells were removed, then blocked in 5% BSA/PBS for 1-2 hr and incubated with primary antibodies overnight at 4 C. Primary antibodies are washed 3-4 times and cells are incubated with secondary antibodies for 1 h at room temperature, washed 2-3 times, incubated with Draq5 and Hoescht for 5 minutes, washed 3 times and mount with ProlongGold™ antifade reagent (Invitrogen). Slides were let to dry overnight, the images were captured with Zeiss confocal microscope;

FIG. 20 shows an in silico analysis of putative sumoylation sites and SUMO-binding sites in human PHB1 protein;

FIG. 21 shows that PHB1 cannot be sumoylated by SUMO-1 in vitro. An in vitro sumoylation assay in the presence of SUMO-1, E1 and E2 enzymes, ATP and purified GST-PHB1 protein indicated that PHB1 cannot be sumoylated in vitro. GST and GST-RanGap1 proteins were used as negative and positive controls respectively. (A) The purified GST and GST fusion proteins were analyzed by SDS-PAGE followed by a Coomassie blue staining. (B) Four times less protein GST were used for the test compared to the fusion proteins. The products of in vitro sumoylation assay were analyzed by immunoblot against PHB1 and RanGap1. The asterisk (*) represents the sumoylated GST-RanGap1;

FIG. 22 shows that PHB1 can bind SUMO-1 proteins via a SBM (SUMO-binding module). (A) Diagram represents various PHB1 constructs generated for the study. Wild type PHB1, a mutant in which the nuclear signal of export was deleted (PHB14NES), or was replaced by a nuclear localization signal (PHB1_NLS), and a mutant where a putative SUMO-binding module was deleted (PHB1_ΔSBM). All PHB1 constructs have triple Flag-tag at the N-terminal. (B) C28/I2 cells were infected with each construct in order to produce stable lines. The nuclear proteins (X-N) and cytoplasmic proteins (X-C) were isolated and analyzed by immunoblot. (C) Co-IP assays with anti-C-myc antibody demonstrated that PHB1 interacts with SUMO-1 through the SBM. SUMO-1 was tagged with c-myc;

FIG. 23 shows that UBC9 expression is increased in knee joint OA cartilage and correlates with disease severity. Left panels are Safranin-O staining and represent the proteoglycan content which decreases with the severity of OA. Right panels represent IHC experiments performed with anti-Ubc9 antibody where staining intensity also correlates with disease progression;

FIG. 24 shows representative immunohistological sections showing UBC9 proteins in normal cartilage (B, D) and knee joint OA (C, E) sections. (A) represents the mean value of UBC9 proteins detected by IHC in superficial and deep zone of normal knee cartilage (n=3) and knee joint OA cartilage sections (n=9). In brief, three sections of each specimen were examined (40× Leica DM R Microscope) from either the superficial zone of the cartilage, scored, and the resulting data integrated as a mean for each specimen. The final results were expressed as the percentage of chondrocytes staining positive for the antigen (cell score) with the maximum score being 100%. Each slide was subjected to evaluation by two observers with >95% degree of agreement. Panels B and C correspond to superficial zones of normal and OA cartilage respectively and panels D and E represent the deep zones of normal and OA cartilage respectively;

FIG. 25 shows that sumoylation stabilizes PHB1 and promotes its nuclear accumulation in U2OS cells. (A) U2OS cells were transfected with the pLPC-3xFlag-PHB1 alone or co-transfected with different components of the sumoylation pathway. (B) The nuclear proteins (X-N) as well as total proteins (X-T) were isolated from cells transfected with pLPC-3xFlag-PHB1, PHB1-NLS or PHB1-ΔSBM in presence or absence of myc-SUMO-1 and UBC9. (C) The cells transfected with the empty vector pLPC-3xFlag or containing PHB1 and PHB1-NLS constructs were treated with the MG132 for 4 hours, then the total proteins were extracted;

FIG. 26 shows human PHB1 mRNA nucleotide (A, obtained from gi|6031190|ref|NM_{—}002634.2) (SEQ ID NO: 1) and amino acid sequence (B, obtained from gi|4505773|ref|NP_{—}002625.1) (SEQ ID NO: 2);

FIG. 27 shows human SUMO-1 precursor mRNA nucleotide (obtained from NCBI Reference Sequence NM_{—}003352.4) (SEQ ID NO: 3) and amino acid sequence (obtained from NCBI Reference Sequence: NP_{—}003343.1) (SEQ ID NO: 4);

FIG. 28 shows human SUMO-2 precursor mRNA nucleotide (obtained from NCBI Reference Sequence NM_{—}006937.3) (SEQ ID NO: 5) and amino acid sequence (obtained from NCBI Reference Sequence: NP_{—}008868.3) (SEQ ID NO: 6);

FIG. 29 shows human SUMO-3 precursor mRNA nucleotide (obtained from NCBI Reference Sequence NM_{—}006936.2) (SEQ ID NO: 7) and amino acid sequence (obtained from NCBI Reference Sequence: NP_{—}008867.2) (SEQ ID NO: 8);

FIG. 30 shows human SUMO-4 precursor mRNA nucleotide (obtained from NCBI Reference Sequence NM_{—}001002255.1) (SEQ ID NO: 9) and amino acid sequence (obtained from NCBI Reference Sequence: NP_{—}001002255.1) (SEQ ID NO: 10); and

FIG. 31 shows human UBC9 mRNA nucleotide (obtained from NCBI Reference Sequence NM_{—}006936.2) (SEQ ID NO: 11) and amino acid sequence (obtained from NCBI Reference Sequence: NP_{—}008867.2) (SEQ ID NO: 12).

### DESCRIPTION OF ILLUSTRATIVE EMBODIMENTS

### Sumoylation and PML Bodies

PML nuclear bodies (PML-NBs) are highly dynamic micro-nuclear structures composed solely of proteins. The main component of PML-NBs is the PML protein (promyelocytic leukemia protein), of which there are seven isoforms in humans (Condemine et al., 2006). So far, the PML-NBs have been associated with several functions such as cell cycle regulation, regulation of gene transcription, response to DNA damage, senescence and apoptosis (Bernardi and Pandolfi, 2007).

The present invention is illustrated in further details by the following non-limiting examples.

### Example 1

### Materials and Methods

### Derivation of Human Articular Chondrocytes

Articular cartilage from OA knee patients was collected, cut into small pieces and washed twice in sterile PBS 1× pH 7.4 (phosphate buffer saline: 0.137 M NaCl, 8.1 mM Na₂HPO₄, 2.7 mM KCl, 1.5 mM KH₂PO₄). For each patient, some pieces were fixed in a solution of paraformaldehyde (PFA) 4% v/v, embedded in paraffin blocks and stored for histological analysis. The pieces of remaining cartilage were incubated for one hour at 37° C. with shaking in D-MEM (Dulbecco's modified Eagle's medium 1×: Wisent Inc., St-Bruno, Quebec, Canada)) containing 10% (v/v) FBS (FCS: Gibco BRL, Burlington, Ontario, Canada), 1% pen-strep and 1 mg/ml pronase (Sigma-Aldrich, Oakville, ON, Canada) and then digested for 4 to 6 hours at 37° C. with stirring presence of 2 mg/ml collagenase (Sigma-Aldrich, Oakville, ON, Canada) diluted in D-MEM supplemented with FBS and pen-strep. The digested tissue was passed through a sieve sterile, and then centrifuged at 215×g for 10 minutes. The pellets of chondrocytes were resuspended in a small volume of culture medium and counting the number and cell viability was performed using the Vi-Cell™ XR Cell Viability analysis (Beckman Coulter: Mississauga, ON, Canada). The cells were then placed in primary culture at high density (2×10⁶ cells) in T-75 flasks and then placed in 10 cm or kneaded Labteks™ according to the desired use. The primary chondrocytes were then either frozen and stored in liquid nitrogen in a solution containing 10% FBS DMSO, or maintained in culture in the first passage for immediate use.

### Cell Lines

All chondrocytes of patients and cell lines, MCF-7 and U2OS were cultured in D-MEM (Dulbecco's modified Eagle's medium 1×: Wisent Inc., St-Bruno, Quebec, Canada) supplemented with 10% (v/v) FBS (FCS: Gibco BRL, Burlington, Ontario, Canada) and 100 units/ml penicillin and 100 μg/ml streptomycin (Gibco BRL, Burlington, Ontario, Canada). C28/I2 cells, a line of human chondrocytes were cultured in medium containing a mixture of D-MEM and F12 (Gibco BRL, Burlington, Ontario, Canada) in a 1:1 ratio, supplemented with 10% FBS (Gibco BRL, Burlington, Ontario, Canada) and penicillin and 100 μg/ml 100 unités/ml streptomycin (Gibco BRL, Burlington, Ontario, Canada). C28/I2 cells were generously provided by the group of Dr. Mary B. Goldring (Cornell University, New York) and MCF-7 cells were provided by the group of Dr. André Tremblay (Research Centre of the CHU Ste-Justine). All cells were grown at 37° C. in an incubator containing 5% CO₂ and 95% air and culture medium was changed every 3 to 4 days.

### Plasmids and Constructs

The different PHB-1 mutants were constructed from a clone of the commercial wild-type prohibitin (Origen) and cloned into the retroviral expression vector PLPC-3xFlag (Calabrese et al., 2009), to mark the protein with a triple Flag epitope in the N-terminal. Four constructs were made with a wild type (PHB-1) and three mutants: PHB1-ΔSBM including a putative binding site of SUMO proteins (residues 76-79) was deleted; PHB1-ΔNES, lacking the signal nuclear export (residues 257-272) and PHB1-NLS, where the NES was replaced by a nuclear localization signal (NLS). The nucleotide sequences of various primers used are shown in Table I below. Plasmids pCDNA3-Myc-SUMO-1, pcDNA-Myc-SUMO-3 and pCDNA-HA-SUMO-2 were provided by the laboratory of Dr. Christopher K. Glass (University of California, San Diego). UBC9 plasmid was provided by the team of Dr. Muriel Aubry (University of Montreal, Montreal). The various plasmids were transformed into DH5α strain of E. coli by heat shock of 45 seconds at 42° C., followed by incubation for 16-18 hours at 37° C. with shaking in 450 ml of medium 2YXT (12.5 g yeast extract, 12.5 g bacto-tryptone/L of water) supplemented with 50 ml of saline (0.17 M KH₂PO₄, 0.72 M K₂HPO₄). The various plasmids were then isolated and purified by cesium gradients.

TABLE I
Nucleotide sequence of the primers used for the
construction of various protein fusions for the
trials of sumoylation in vitro and for the
overexpression of proteins. S: sense,
AS: antisense.

| Constructs | Plasmids | PRIMERS |
|---|---|---|
| PHB-1 | pLPC-3xFlag | S: 5′ GCGAATTCTGCTGCCAAAGTG |
| | | TTTGAGTCCATTGGC 3′ |
| | | (SEQ ID NO: *) |
| | | AS: 5′ GCCTCGAGTCACTGGGGCAG |
| | | CTGAGGA 3′ |
| | | (SEQ ID NO: *) |
| PHB-1-NLS | pLPC-3xFlag | AS: 5′ GCCTCGAGTCAGCCCACTTT |
| | | GCGCTTTTTCTTGGGG |
| | | (SEQ ID NO: *) |
| | | TTCCGAGAGCGTGAGAGCTGGTA 3′ |
| | | (SEQ ID NO: *) |
| PHB-1-ΔNES | pLPC-3xFlag | AS: 5′ GCCTCGAGTCAGTTCCGAGA |
| | | GCGTGAGAGCTGGTA 3′ |
| | | (SEQ ID NO: *) |
| PHB-1-ΔSBM | pLPC-3xFlag | S: 5′ CCACGTAATACTGGTAGCAAA |
| | | GATTTACAGAATGTC 3′ |
| | | (SEQ ID NO: *) |
| | | AS: 5′ GCTACCAGTATTACGTGGTC |
| | | GAGAACGGCAGTCA 3′ |
| | | (SEQ ID NO: *) |
| GST-RanGap1 | pGEX-5X-3 | S: 5′ ACGGATCCCCGCCTCGGAAGA |
| | | CATTGCCAAG 3′ |
| | | (SEQ ID NO: *) |
| | | AS: 5′ ATGAATTCGACCTTGTACAG |
| | | CGTCTGCAGCAG 3′ |
| | | (SEQ ID NO: *) |

### Immunohistochemistry

The cartilage slices, mounted on SuperFrost™ slides (Fisher Scientific, Hampton, N.H., USA) were deparaffinized by soaking slides in three successive baths of toluene, rehydrated in four baths of alcohol at 100%, 90%, 70% and 50%, and washed in a water bath and a 1×PBS pH 7.4 bath, at 5 minutes per bath. The slides were then heated 20 minutes at 65° C. in a solution of sodium citrate, 0.01 mM, pH 6.0, then washed 5 minutes with PBS 1×. The slides were then incubated for 30 minutes in a solution of 1×PBS containing 0.3% Triton™ and washed 3 times 5 minutes with PBS 1×. After a 30 min. incubation in methanol containing 2% H₂O₂, slides were placed in a humidity chamber and the sections were incubated for 1 hour at room temperature in a blocking solution containing normal horse serum (ABC Vectasin™ kit, Vector Laboratories, Curlingame, Calif.). The sections were then incubated overnight in blocking solution containing primary antibody (anti-PHB1: Ab-2, Neomarker), washed 3 times in 1×PBS, then incubated 45 minutes in the presence of biotinylated secondary antibody diluted in blocking solution, and washed again three times with PBS 1×. After incubation with avidin-biotin complex (ABC Vectasin™ kit, Vector Laboratories, Curlingame, Calif.), the labeling was revealed using the system diaminobenzidine (DAB) (Dako Diagnostics Canada Inc., Mississauga, ON, Canada) according to the manufacturer's instructions, giving a brownish color to the expressed protein. The sections were then stained with Harris hematoxylin (Fisher Scientific, Hampton, N.H., USA) and mounted with a coverslip using Permount® ®(Fisher Scientific, Hampton, N.H., USA).

### Extraction of Total Protein

One petri dish of confluent cells per condition were washed twice in cold 1×PBS, then harvested and lysed in lysis buffer (20 mM Tris-HCl pH 7.5, 150 mM NaCl, 1 mM EDTA, 1 mM EGTA, 1% Triton™ X-100, 2.5 mM sodium pyrophosphate, 1 mM β-glycerophosphate) containing in addition to a cocktail of protease inhibitors 1× (Roche, Indianapolis, Ind., United Kingdom) and 25 mM NEM (Sigma-Aldrich, Oakville, ON, Canada). After 30 to 60 minutes of incubation at 4° C. with shaking, the protein lysates were collected after centrifugation for 15 minutes at 11,200×g.

### Separation of Nuclear and Cytoplasmic Proteins

Two or three petri dishes of confluent cells (5×10⁶ cells/petri dish) by condition were washed twice in 1×PBS (10.1 mM Na₂HPO₄, 1.8 mM KH₂PO₄, 2.7 mM KCl, 137 mM NaCl) cold, scraped and transferred into 1.5 ml tubes. After a centrifugation at 100×g for 5 minutes, the cell pellets were resuspended in 300 μl of hypotonic lysis buffer (10 mM HEPES pH 7.9, 1.5 mM MgCl₂, 10 mM KCl, 1% NP-40, 0.5 mM DTT) supplemented with a cocktail 1× of protease inhibitors (Roche, Indianapolis, Ind., United Kingdom) and 25 mM NEM (Sigma-Aldrich, Oakville, ON, Canada), incubated on ice for 25 minutes by vortexing every 3 to 4 minutes. The lysates were centrifuged at 4° C. for 5 minutes at 1200×g to obtain a pellet containing the cell nuclei. The supernatants containing the cytoplasmic proteins were transferred to new 1.5 ml tubes and recentrifuged a second time at 4° C. for 10 minutes at 1200×g to remove remaining debris and minimize contamination by nuclear proteins. The supernatants were transferred back into new 1.5 ml tubes. The nuclei pellets were resuspended in 8 ml of nuclear lysis buffer (50 mM Tris-HCl pH 7.6, 2 mM EDTA, 2 mM EGTA, 1 mM DTT, protease inhibitor cocktail 1× (Roche, Indianapolis, Ind., United Kingdom), 25 mM NEM (Sigma-Aldrich, Oakville, ON, Canada) containing 0.1% Triton™ X-100 and placed on 2 ml of sucrose cushion (nuclear lysis buffer containing 30% w/v sucrose) in tubes of 15 ml. The samples were then centrifuged at 4° C. for 50 minutes at 3500×g in a Sorvall™ Legend RT centrifuge. The buffer was decanted to leave the bottom of the tubes that the pellets of nuclei purified which were resuspended in 50 to 100 μl 4× Laemlli buffer (0.52 M Tris-HCl pH 6.8, 6.85% SDS, 3.3% 3-mercaptoethanol, 20% glycerol) and boiled for 5 minutes. After quantification of proteins by a Bradford assay (Bio-Rad, Hercules, Calif., United States), 50 mg of cytoplasmic and nuclear proteins were separated by SDS-PAGE and analyzed by Western blot.

### Immuno/Co-Immunoprecipitation Assays

U2OS cells were transfected with pCMV4-Myc-sumo1 in the presence of PLPC-3xFlag-PHB-1 or PLPC-3xFlag-PHB1-ΔSBM (15 g total DNA) by using calcium phosphate precipitation. The culture medium was changed 24 hours post-transfection and cells were harvested 48 hours after transfection. The cells were washed twice in cold 1×PBS, then harvested and lysed in lysis buffer (20 mM Tris-HCl pH 7.5, 150 mM NaCl, 1 mM EDTA, 1 mM EGTA, 1% Triton™ X-100, 2.5 mM sodium pyrophosphate, 1 mM 3-glycerophosphate) containing in addition to a cocktail of protease inhibitors 1× (Roche, Indianapolis, Ind., United Kingdom) and 25 mM NEM (Sigma-Aldrich, Oakville, ON, Canada). After 30 to 60 minutes of incubation at 4° C. with shaking, the protein lysates were harvested by centrifugation for 20 min at 11,200×g. The immunoprecipitations were performed overnight at 4° C. in the presence of 1 to 2 mg of total protein and the primary antibody. The following antibodies were used: anti-PHB1 (N-20, Santa Cruz), anti-c-myc (MAB8865, Millipore). The immunoprecipitates were collected by following a 1-hour incubation at 4° C. in the presence of protein A/G Sepharose™ (Amersham Biosciences Corp., Qc, Canada) and washed 3 times with the lysis buffer, 1 time in 1×PBS and 1 time with water. The precipitates were eluted in 70 μl 3× Laemlli buffer, boiled for 5 minutes and 35 μl were used for analysis by Western blot.

### GST Pull-Down

Nucleic acid encoding PHB-1 and RanGap1 proteins were first cloned into the vector pGEX-5X-3. The different GST-fusion proteins were produced in E. coli strain BL21. Each of the plasmids, including the empty vector, was transformed by heat shock of 45 seconds at 42° C. Bacteria containing each of the plasmids were grown at 37° C. in 400 ml of 2YXT medium (16 g/L tryptone, 10 g/L yeast extract, 5 g/L NaCl) to an optical density of 0.8 at 600 nm, then induced in the presence of 0.4 mM IPTG for 4 hours at 30° C.

The bacterial pellets from a 250 ml culture were resuspended in 3 ml of STE buffer (10 mM Tris-HCl pH 8, 1 mM EDTA, 100 mM NaCl) supplemented with DTT (5 mM), PMSF (1 mM) and a cocktail of protease inhibitors (Roche, Indianapolis, Ind., United Kingdom). Then, 1 mg/ml of lysozyme (Sigma-Aldrich, Oakville, ON, Canada) was added. After a 30-45 minutes incubation on ice, 1.5% sarcosyl (Sigma-Aldrich, Oakville, ON, Canada) was added and sonication was performed (5 times, 10 seconds per tube). Cell lysates were then transferred into 2 ml tubes and centrifuged 10 minutes at 11,200×g at 4° C. The supernatants were transferred to new tubes and 120 μl of 2 ml of glutathione beads/50% Sepharose™ (Amersham Biosciences Corp., Qc, Canada) were added to each tube. After incubation with agitation at 4° C. for 2 hours, the beads were washed two times in NETN buffer (10 mM Tris-HCl pH 8, 1 mM EDTA, 100 mM NaCl, 0.5% NP-40), once in NETN buffer 500 mM NaCl and again once in the NETN buffer. The beads were finally resuspended in an equal volume of 1×PBS supplemented with protease inhibitors (Roche, Indianapolis, Ind., United Kingdom). 5 μl of beads were then analyzed by SDS-PAGE followed by staining with Coomassie blue.

### Sumoylation Assays

GST-PHB1 fusion proteins purified by GST pull-down were used as substrate for the testing of sumoylation by SUMO-1. GST and GST-RanGap1 proteins were used as negative and positive controls, respectively. Each reaction was performed in a total volume of 20 μl in a reaction buffer containing 20 mM Hepes pH 7.5 and 5 mM MgCl² in the presence of 7.5 mg/ml of E1 enzyme, 50 μg/ml of E2 enzyme, 50 μg/ml of Sumo-1 and 20 mM ATP for 1 hour at 37° C. All reagents were obtained commercially (LAE Biotech International) and used according to the manufacturer's instructions. For each reaction, 5 μl were then separated on SDS-PAGE gel and analyzed by Western blot.

### Western Blots

All protein extracts were separated by SDS-PAGE on acrylamide gels using the Mini-Protean™ II (BioRad, Hercules, Calif.). The gels consisted of a stacking gel consisting of 4% acrylamide (v/v) in 0.5 M Tris buffer pH 6.8 and a resolving gel containing between 8 and 12.5% of acrylamide (v/v) and 1.5 M Tris buffer pH 8.8. The migration of proteins was carried out at room temperature at a voltage of 120 volts. The proteins were then transferred to PVDF membranes (Millipore) for 90 minutes at a voltage of 100 volts. Once the transfer was complete, the membranes were pretreated by incubation for a few seconds in methanol, followed by a one hour incubation in blocking solution (1×PBS, 0.02% Tween™-20, 10% milk fat-free). Following three 15-minute wash in PBST (1×PBS, 0.02% Tween™-20), the membranes were incubated overnight at 4° C. in the presence of the primary antibody (Table II) diluted in a solution of PBST containing 3% BSA (bovine serum albumin: Bioshop) and 0.02% sodium azide. The next day the membranes were incubated in the presence of secondary antibodies coupled to peroxidase (Thermo Scientific, Rockford, United Kingdom) diluted in PBST containing 5% non-fat milk for one hour at room temperature. After a 1-hour wash in PBST, signals were revealed using the ECL reagent (enhanced chemiluminescence substrate: PerkinElmer, Watlham, Mass., United Kingdom) and detected on an autoradiography film (Amersham Biosciences Corp., QC, Canada). To make a second immunoblotting on the same membrane, the membranes were incubated in a solution containing 25 mM glycine pH 2.0 and 1% SDS (sodium dodecyl sulfate) for 45 minutes at room temperature with agitation to remove antibodies already present. The same steps were then repeated from the blocking step.

TABLE II
Antibodies used for Western blots

| Primary antibodies | Origin | Company |
|---|---|---|
| anti-PHB-1 (Ab-1) | monoclonal, mouse | Lab vision |
| anti-Sumo-1 (#4930) | polyclonal, rabbit | Cell Signaling |
| anti-PanSumo (#AP1299a) | polyclonal, rabbit | Abgent |
| anti-Lamine A/C (#2032) | polyclonal, rabbit | Cell signaling |
| anti-GAPDH (V-18) | polyclonal, goat | Santa cruz |
| anti-Flag (M2) | monoclonal, mouse | Sigma |
| anti-RanGap1 | monoclonal, mouse | Zymed |
| anti-GST (M1) | polyclonal, rabbit | Millipore |

### Immunofluorescence

Chondrocytes of OA patients and healthy subjects at the first passage were grown in 8-well LabTek at a rate of 10 000 cells per well. After 24 to 48 hours of incubation, the cells were washed two times in 1×PBS, fixed in a solution of 3.7% paraformaldehyde, and permeabilized in 1×PBS containing 0.1% Triton™ X-100 for 10 minutes. The cells were then incubated for 30 minutes in blocking solution (PBSA) containing 1×PBS supplemented with 1% bovine serum albumin (BSA: BioShop, Burlington, ON, Canada). Subsequently, the cells were incubated with primary antibodies (Table III below) diluted in PBSA for 2 hours at 37° C. The secondary antibodies (Alexa Fluor™, Invitrogen, Eugene, Oreg., United States) diluted in PBSA were then applied for 1 hour at 37° C. After 3 washes in 1×PBS, the slides were mounted using an adhesive containing DAPI (Prolong Gold™: Invitrogen, Eugene, Oreg., United States) and then observed by confocal microscopy.

TABLE III
Antibodies used for immunofluorescence

| Primary antibodies | Origin | Company |
|---|---|---|
| anti-PHB1 (Ab-1) | monoclonal, mice | Lab vision |
| anti-Sumo-1 (A21C7) | monoclonal, rabbit | Cell Signaling |
| anti-Sumo-2/3 (18H8) | monoclonal, rabbit | Cell Signaling |
| anti-PML (PG-M3) | monoclonal, mice | Santa Cruz |
| Anti-PHB1 (#2413-1) | monoclonal, rabbit | Epitomics |
| Anti-PHB1 (#II-14-10) | monoclonal, mice | NeoMarkers |

### Confocal Microscopy

The slides were observed using a Zeiss™ LSM 510 Meta confocal microscope (Zeiss Canada, Toronto, ON, Canada) at a magnification of 630×. The images were then analyzed using the Zeiss® ®LSM image browser software.

### Example 2

### PHB1 Levels in Plasma Samples Obtained from Osteoarthritis Patients and Age-Matched Healthy Subjects

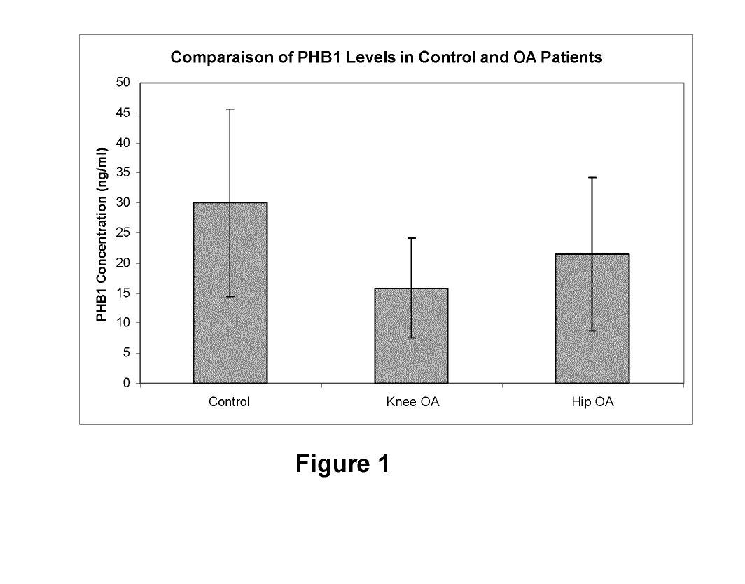

FIG. 1 depicts results from experiments performed on blood samples (plasma) from knee (n=43, demographic characteristics depicted in Table IV below) and hip (n=44, demographic characteristics depicted in Table V below) osteoarthritis patients and age-matched healthy subjects (n=31, demographic characteristics depicted in Table VI), which show that mean plasma levels of the pitx1 repressor protein PHB1 are significantly lower in osteoarthritis patients. The source of circulating PHB1 may be PHB1 shed from the plasma membrane (Mielenz D et al. J Immunol 2005; 174(6):3508-3517), or released from adipocytes and possibly other cells in lipid droplets (Brasaemle D L et al. J Biol Chem 2004; 279(45):46835-46842). The Kellgren-Lawrence (KL) radiographic score also presented and differentiates the severity of OA in Table V (from 1 to 4, wherein 4 corresponds to the most severe form of OA; N/A=data not available). Early stage of OA is defined as patients exhibiting a KL score ≦2, while late stage OA patients was defined as those exhibiting a KL score ≧3.

TABLE IV
Demographic characteristics of recruited subjects for HIP.
Patient Random

| ID | Gender | Age (Years) | BMI |
|---|---|---|---|
| OA-1 | Female | 77 | 31.05 |
| OA-3 | Female | 57 | 32.46 |
| OA-5 | Male | 76 | 28.98 |
| OA-6 | Female | 69 | 28.54 |
| OA-9 | Female | 76 | 21.87 |
| OA-10 | Female | 55 | 22.98 |
| OA-20 | Female | 66 | 28.65 |
| OA-21 | Male | 45 | NA |
| OA-24 | Male | 50 | 32.55 |
| OA-30 | Male | 79 | 30.83 |
| OA-31 | Female | 71 | 29.09 |
| OA-32 | Female | 57 | 25.91 |
| OA-35 | Female | 55 | 27.4 |
| OA-38 | Female | 77 | 29.51 |
| OA-39 | Male | 60 | 35.98 |
| OA-40 | Male | 70 | 29.76 |
| OA-42 | Female | 66 | 33.17 |
| OA-43 | Male | 69 | 21.79 |
| OA-44 | Male | 66 | 58.13 |
| OA-46 | Male | 48 | 29.04 |
| OA-51 | Male | 59 | 27.04 |
| OA-53 | Female | 58 | 21.63 |
| OA-54 | Female | 37 | 16.13 |
| OA-72 | Male | 62 | 28.08 |
| OA-75 | Male | 52 | 28.68 |
| OA-76 | Male | 63 | 33.19 |
| OA-77 | Female | 55 | 41.23 |
| OA-78 | Female | 73 | 31.58 |
| OA-79 | Female | 76 | 22.03 |
| OA-80 | Male | 47 | 34.81 |
| OA-93 | Female | 75 | 25.53 |
| OA-94 | Female | 77 | 34.88 |
| OA-96 | Female | 66 | 27.47 |
| OA-97 | Male | 40 | 31.43 |
| OA-102 | Male | 48 | 28.08 |
| OA-110 | Female | 87 | 26.84 |
| OA-111 | Male | 82 | 31.8 |
| OA-118 | Male | 64 | 28.7 |
| OA-120 | Male | 49 | 25.28 |
| OA-121 | Male | 56 | 30.08 |
| OA-122 | Male | 56 | 35.92 |
| OA-123 | Female | 55 | 20.7 |
| OA-127 | Male | 11 | 28.66 |
| OA-128 | Female | 13 | 32.87 |
| OA-285 | Female | 13 | 23.04 |

TABLE V
Demographic characteristics of recruited subjects-Knee
Patient Random

Characteristic

| ID | Gender | Age (Years) | KL Score | BMI |
|---|---|---|---|---|
| OA-2 | Female | 77 | 2 | 25.51 |
| OA-11 | Female | 54 | 3 | 41.86 |
| OA-14 | Female | 76 | 3 | 41.11 |
| OA-16 | Male | 59 | 4 | 33.63 |
| OA-26 | Female | 75 | 4 | 40.64 |
| OA-33 | Male | 78 | 1 | 25.77 |
| OA-37 | Female | 65 | 4 | 33.67 |
| OA-57 | Female | 77 | 2 | 28.51 |
| OA-59 | Male | 57 | 1 | 34.08 |
| OA-64 | Male | 74 | 3 | 36.5 |
| OA-66 | Female | 72 | 1 | 29.68 |
| OA-95 | Male | 66 | 1 | 36.06 |
| OA-103 | Female | 72 | 2 | 28.12 |
| OA-104 | Female | 70 | 3 | 29.39 |
| OA-105 | Female | 68 | 3 | 38.39 |
| OA-106 | Female | 81 | 3 | 27.94 |
| OA-112 | Male | 70 | 2 | 33.7 |
| OA-134 | Female | 72 | 2 | 35.49 |
| OA-139 | Female | 69 | 3 | 36.26 |
| OA-140 | Female | 78 | 4 | 23.82 |
| OA-152 | Male | 69 | NA | 22.85 |
| OA-153 | Male | 53 | 1 | 29.38 |
| OA-155 | Female | 74 | 4 | 31.14 |
| OA-158 | Male | 58 | 1 | 29.06 |
| OA-159 | Male | 67 | 2 | 36.93 |
| OA-165 | Male | 65 | 3 | 30 |
| OA-186 | Female | 59 | 2 | 30.11 |
| OA-187 | Female | 66 | 1 | 32.03 |
| OA-188 | Male | 58 | 3 | 32.17 |
| OA-206 | Female | 61 | 3 | 34.44 |
| OA-208 | Female | 58 | 1 | 27.58 |
| OA-209 | Female | 70 | 2 | 29.39 |
| OA-210 | Female | 77 | 2 | 23.61 |
| OA-211 | Male | 64 | 2 | 29.51 |
| OA-212 | Female | 74 | 3 | 22.65 |
| OA-223 | Female | 66 | 2 | 43 |
| OA-225 | Male | 79 | 3 | 25.61 |
| OA-227 | Male | 54 | 3 | 37.87 |
| OA-228 | Female | 59 | 4 | 35.49 |
| OA-239 | Female | 80 | 4 | 29.03 |
| OA-266 | Female | 47 | 1 | 38.08 |
| OA-267 | Male | 55 | 2 | 30.38 |
| OA-281 | Female | 78 | 3 | 26.67 |

TABLE VI
Demographic characteristics of recruited subjects-Control
Patient Random

| ID | Gender | Age (Years) | BMI |
|---|---|---|---|
| CTRL-4 | Male | 61 | 31.67 |
| CTRL-7 | Female | 46 | 21.33 |
| CTRL-8 | Male | 32 | 26.87 |
| CTRL-23 | Female | 19 | 23.53 |
| CTRL-27 | Female | 51 | 28.12 |
| CTRL-60 | Female | 19 | 21.29 |
| CTRL-61 | Female | 49 | 23.87 |
| CTRL-68 | Male | 36 | 25.53 |
| CTRL-69 | Male | 47 | 28.36 |
| CTRL-71 | Female | 37 | 23.24 |
| CTRL-73 | Female | 49 | 17.84 |
| CTRL-74 | Female | 43 | 22.03 |
| CTRL-83 | Female | 52 | 22.71 |
| CTRL-84 | Male | 46 | 24.03 |
| CTRL-86 | Female | 52 | 19.43 |
| CTRL-87 | Female | 49 | 21.9 |
| CTRL-90 | Female | 38 | 23.04 |
| CTRL-91 | Female | 37 | NA |
| CTRL-92 | Male | 25 | NA |
| CTRL-98 | Female | 57 | 21.09 |
| CTRL-99 | Female | 32 | NA |
| CTRL-100 | Male | 50 | NA |
| CTRL-108 | Female | 55 | 28.28 |
| CTRL-109 | Female | 42 | NA |
| CTRL-114 | Female | 65 | NA |
| CTRL-131 | Female | 24 | NA |
| CTRL-136 | Female | 77 | 24.43 |
| CTRL-137 | Female | 41 | 24 |
| CTRL-143 | Male | 18 | NA |
| CTRL-316 | Male | 24 | NA |
| CTRL-329 | Male | 32 | NA |

### Example 3

### PHB1 Levels in Plasma Samples Obtained from Osteoarthritis Patients, Men and Women, Healthy Subjects and Subjects Having Rheumatoid Arthritis

Plasmatic PHB1 levels were determined in a group of 231 patients. Plasma was isolated from peripheral blood by centrifugation and frozen at −80 C until analysed. ELISA analysis was performed as per vendor protocol (Uscnk (www.uscnk.us), Prohibitin kit. Protocol manual 7^{th} edition revised in November 2011)

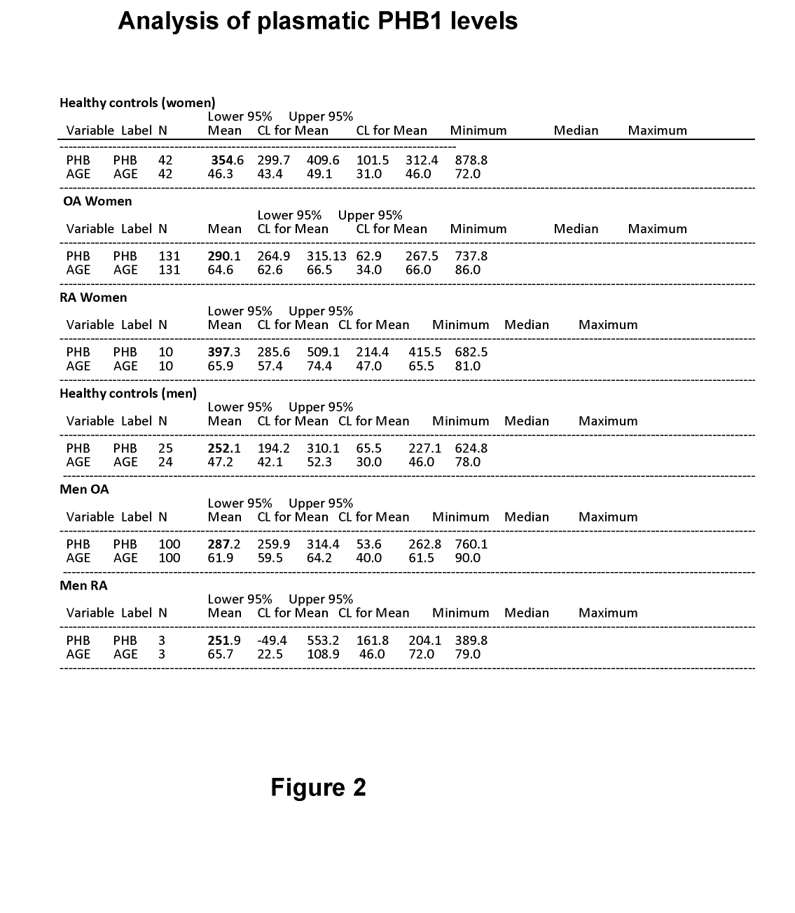

Results are presented in FIG. 2 and presents the values of PHB1 plasma levels by sex and health group. Plasma levels of PHB1 was obtained using Uscnk ELISA kit for PHB1. Analysis of the values was performed using Proc Logistic in SAS v9.2. Procedure Proc means was used to calculate the average (mean), minimum, maximum, median, and the 95% confidence interval of the PHB1 values for healthy subjects, OA and RA for man and women. FIG. 2 shows a statistically significant decrease in circulating PHB1 of OA patients as compared to control subjects.

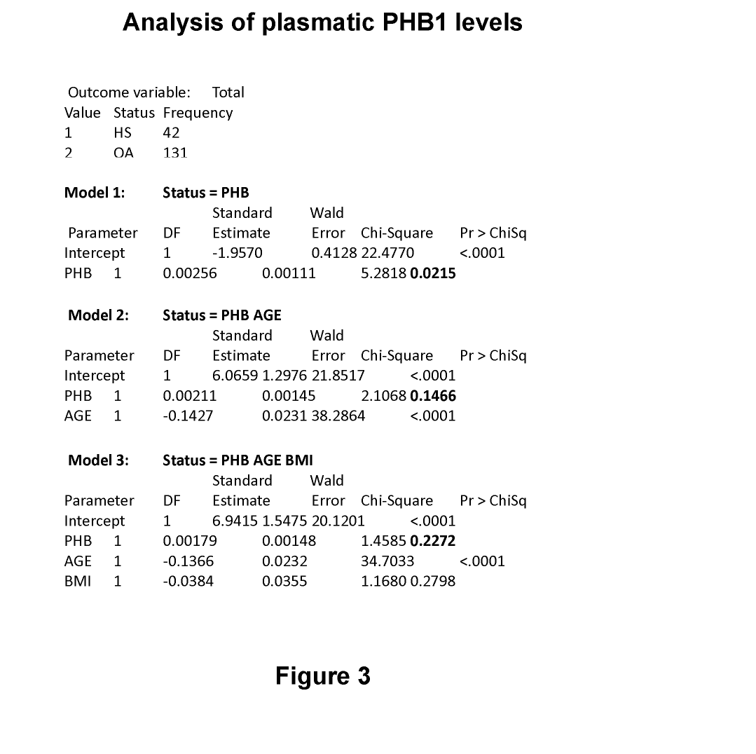

Statistical analysis system (SAS) is is a software made by the SAS institute: http://www.sas.com/company/about/history.html. SAS output analysis were performed using data for women only as an interaction exist between levels of PHB1 and sex. In the logistic model used, the outcome is OA or Healthy status and the predictor is PHB alone or in combination of co-variate(s) (see FIG. 3). These analyses were performed with the Proc Logistic procedure in SAS v9.2. Logistic models were used to analyse the significance of PHB1 plasmatic levels on the prediction of OA vs healthy status. These analyses were performed in a step-wise manner. First with PHB1 levels alone and then in presence of covariates (age and BMI) as these two covariates that are also associated with the occurrence of OA. Reported is the p values for PHB1 and covariates. The fact that the results obtained with OA men did not show a statistically significant difference vs. control subjects may be explained by the small sample size and/or that the origin of the arthrosis was secondary (trauma) rather than primary (genetic predisposition).

### Example 4

### Distribution of PHB1 in the Cytosol and Nucleus of Lymphocytes from OA Patients

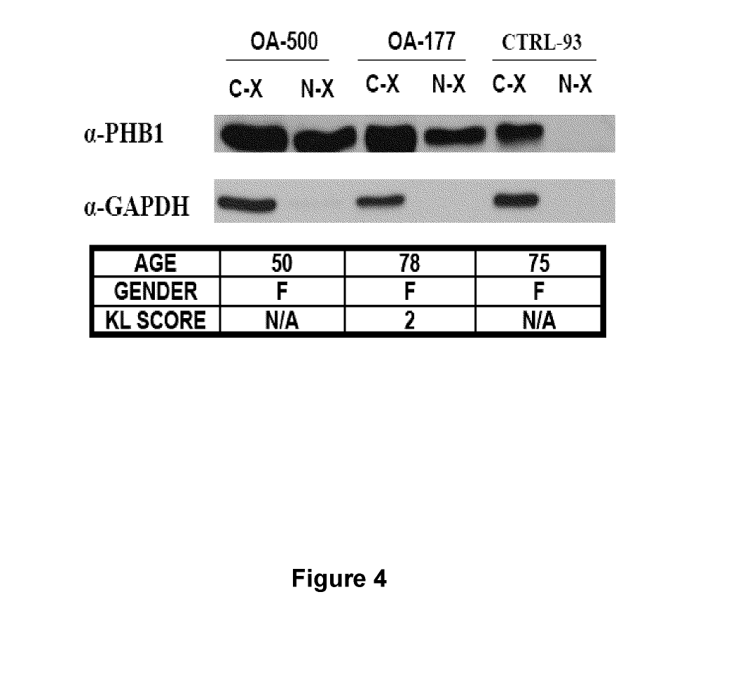

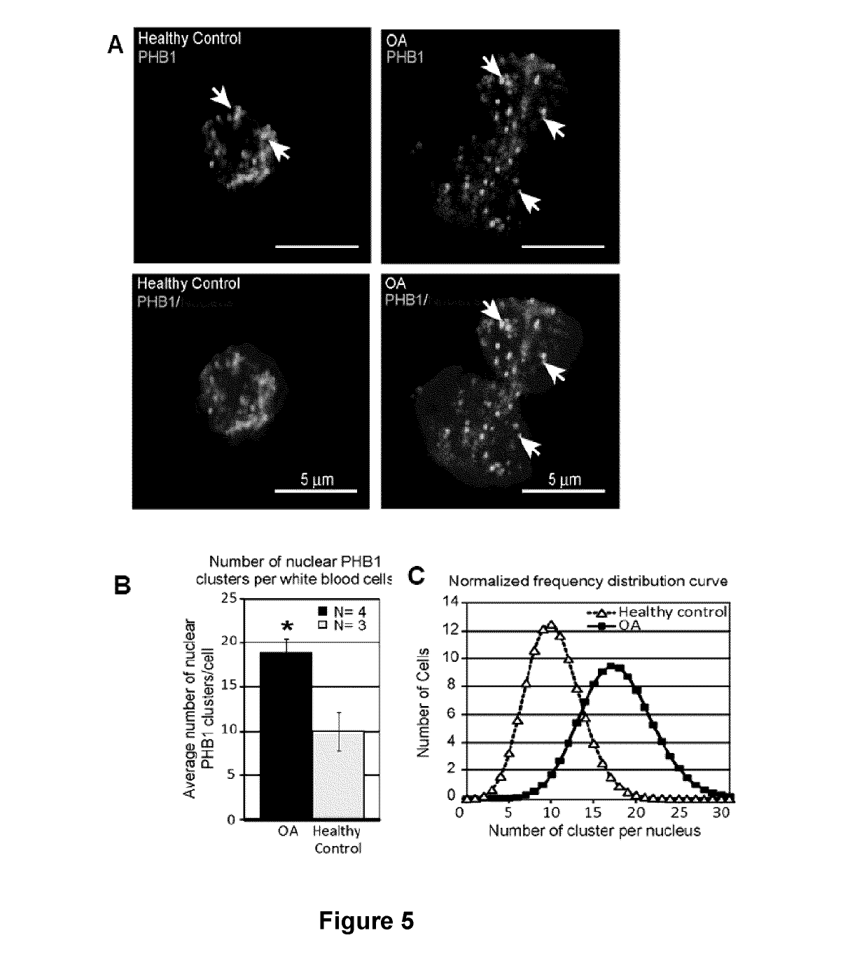

FIGS. 4 and 5 depict results from Western blot (FIG. 4) and immunofluorescence (FIG. 5) experiments that show that PHB1 is found at higher levels in the nucleus of human lymphocytes from two OA patients, relative to lymphocytes from a control subject. The KL radiographic score was also used to differentiate the severity of OA. Early stage of OA was defined as patients exhibiting a KL score ≦2, while late stage OA patients was defined as those exhibiting a KL score ≧3 (FIG. 4). FIG. 5 shows the accumulation of PHB1 in clusters in lymphocyte nucleus. In support with an increase in nuclear PHB1 levels (FIG. 4), an increase in the number of nuclear agglomeration in OA cells indicates an increase quantity of nuclear PHB1 in these cells.

### Example 5

### PHB1 Level is Higher in Leucocytes Nuclei from OA Patients

Leucocytes (lymphocytes and monocytes) obtained from healthy subjects or OA patients were isolated by Ficoll™ gradient, centrifuged onto eight-wells chamber slides (coated with poly-D-Lysine) and immunostained for PHB1. Nuclei were counterstained with DragS and Hoechst. Confocal images of PHB1 staining were obtained by adjusting the focal plane (less than 1 micron thick) at the center of the nuclear signal. For each sample, gain detector (equivalent to exposure time) for PHB1 signal was adjusted such that only a few pixels of the brightest cells were saturated (this was done using the “palette” function of the image acquisition software). The intensity of the PHB1 signal was measured indirectly by adjusting settings of the microscope camera to generate images with the same saturation levels. Then the settings used were compared between healthy subjects and OA patients.

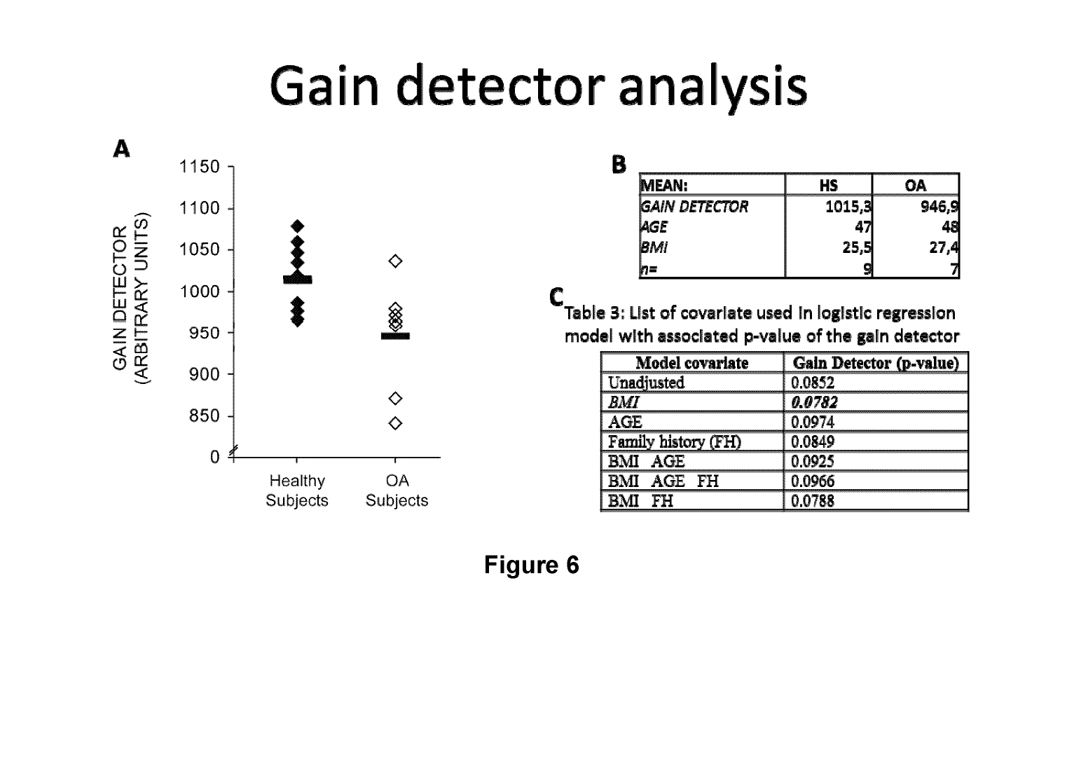

For this approach 9 controls and 7 OA patients matched for age, BMI and sex (all women) were used. Data is represented in FIG. 6. As shown in FIG. 6, PHB1 signal is increased in leucocytes from OA patients.

The statistical analyses were performed using a logistic model (using SAS v9.2) for which the subject condition (OA vs healthy) was predicted from the values of gain detector adjusted with the covariate listed in Table 3 of FIG. 6. Since both the mean age and the percentage of subject with family history are very similar in both groups, these two variables could be omitted in the final analysis to obtain the simplest model in which only BMI is included as a covariate. No interactions were observed among the listed variable. Using that simple model, the significance of the gain detector is 0.0782, which is close to significance.

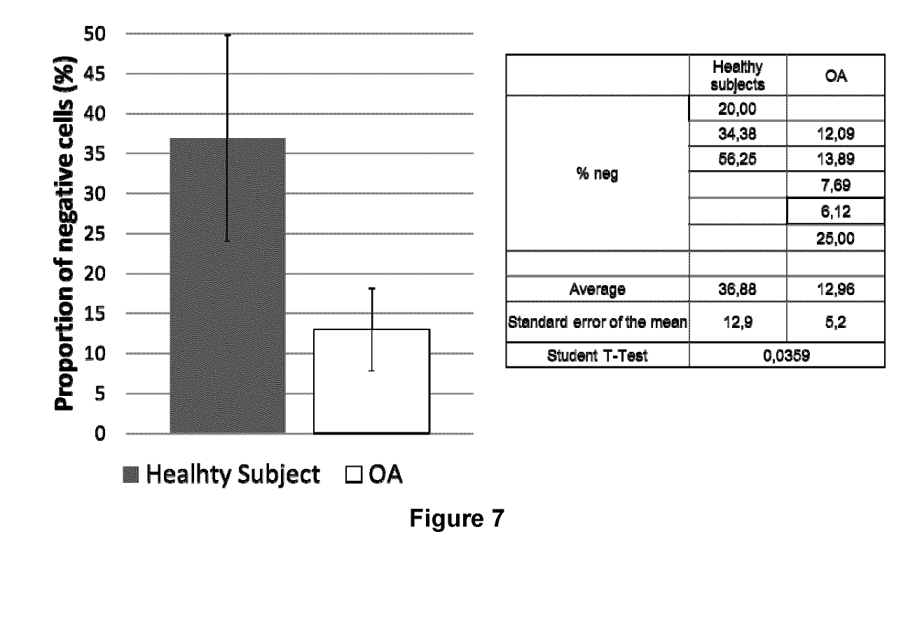

In addition, the percentage of leucocytes nuclei expressing low levels of PHB1 was determined (FIG. 7). For this set of experiment, the gain detector was kept constant and the percentage of cell with non-detectable signal for PHB was calculated out of a minimum of 20 cells (3 healthy subjects and 5 OA patients were used in this analysis).

The results presented in FIGS. 6 and 7 suggest that nuclear levels of PHB1 are lower in healthy subjects than in OA diagnosed patients.

### Example 6

### The Number of Nuclear PHB1 Agglomerates is Similar in Control and Osteoarthritis Patients

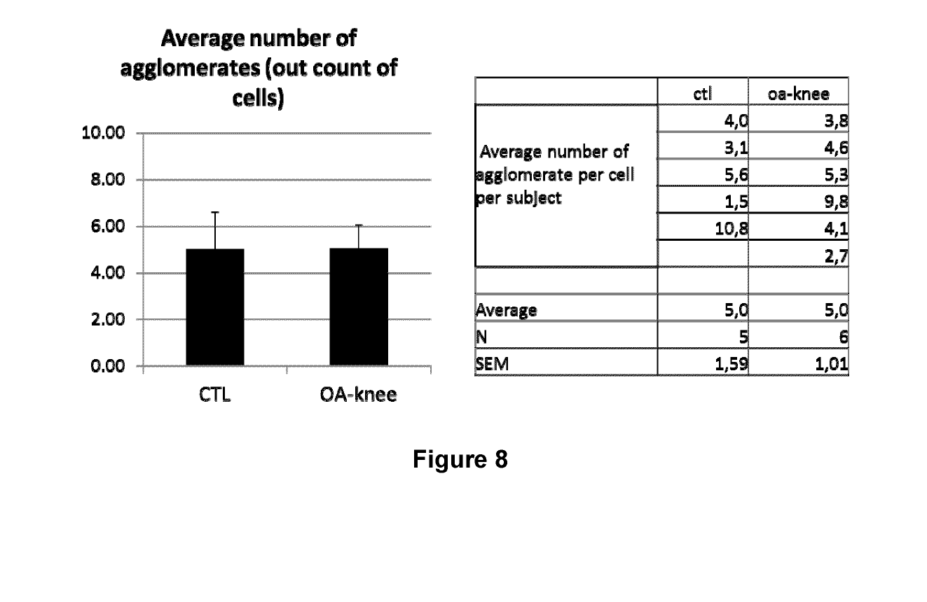

FIG. 8 shows the analysis of the average number of nuclear PHB1 agglomerates in leucocytes nucleus in control subjects (n=5) and OA subjects (n=6).

For these analyses the number of nuclear agglomerates was manually counted for a minimum of 20 cells per patient from confocal images.

FIG. 8 shows that there is no significant difference in the average number of PHB1 agglomerates in leucocytes from control and OA subjects.

### Example 7

### PHB1 Level is Higher in Chondrocytes Nuclei from OA Patients

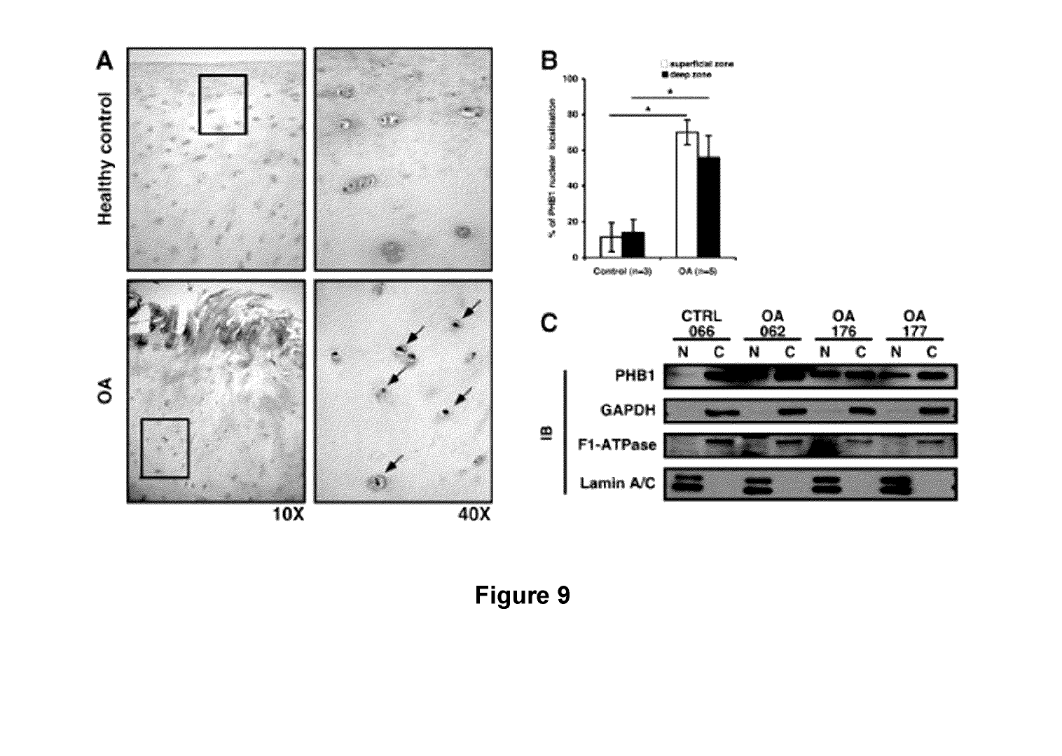

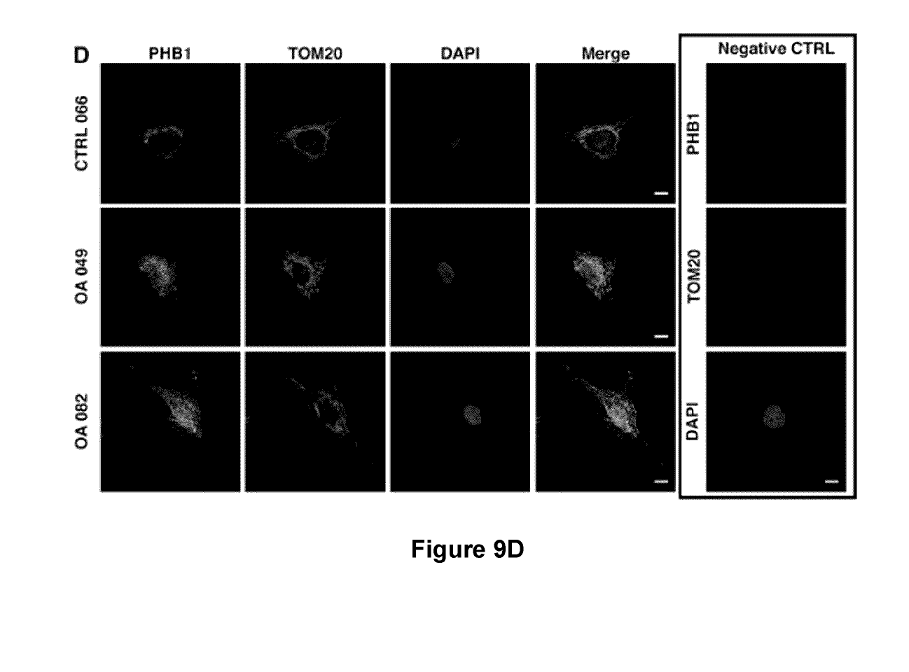

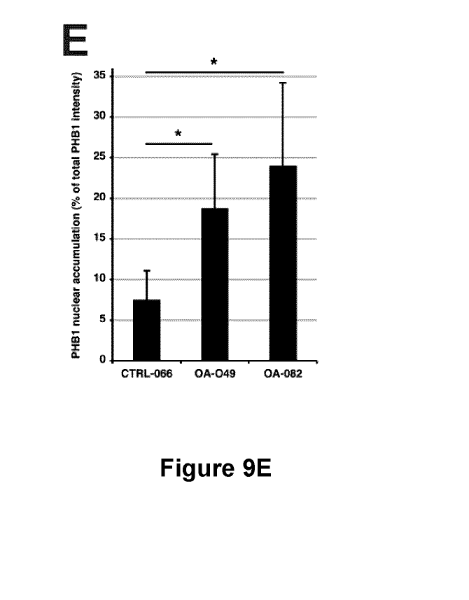

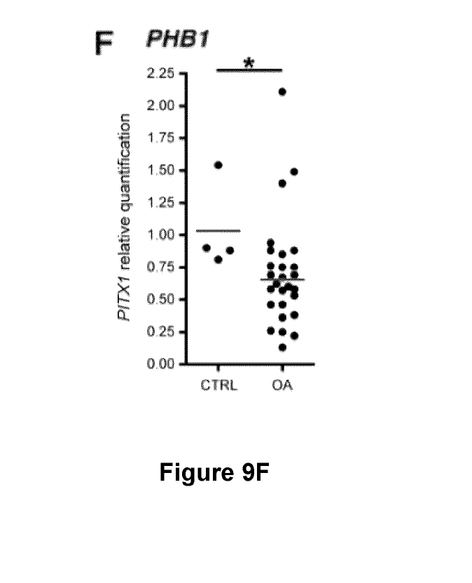

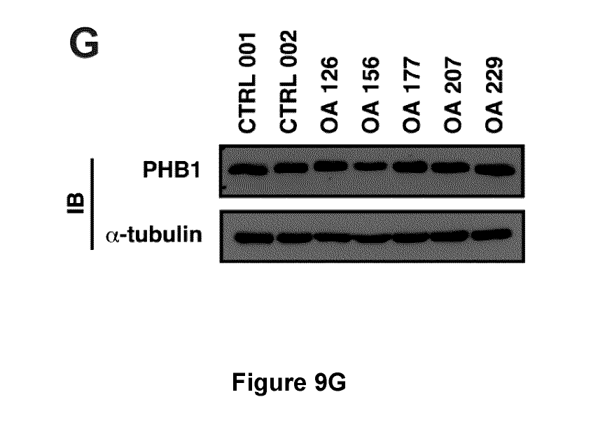

PHB1 expression levels were compared with its cellular localization in normal and OA articular chondrocytes by IHC assays (FIG. 9A). This experiment revealed a strong positive signal for PHB1 in the nuclei in a majority of OA patients, which correlates with Pitx1 repression. Indeed, PHB1 mostly accumulated in nuclei from OA chondrocytes (60% of total PHB1 in the nuclei), whereas it was mainly present in the cytoplasm of control chondrocytes (10% in the nuclei), and this in both superficial and deep zones of articular cartilage (FIG. 9B).

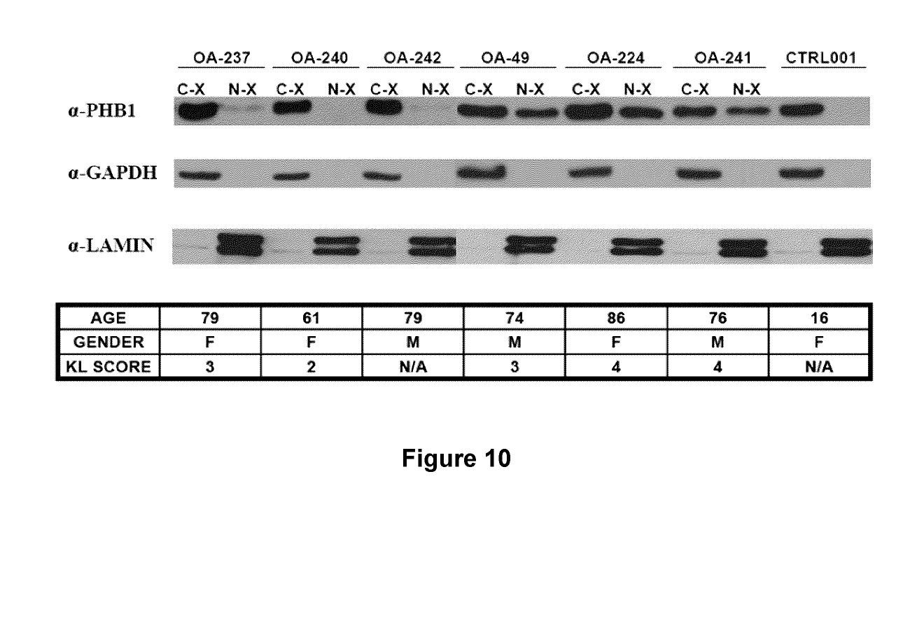

To confirm these results, subcellular localisation of PHB1 was investigated by isolation of nuclear and cytosolic extracts using a saccharose gradient and verified by measuring through immunoblotting the relative presence of PHB1 in the two cellular fractions isolated from control and OA primary chondrocytes cultures. PHB1 was detected in both fractions in OA chondrocytes whereas in control chondrocytes, it was detectable in the cytoplasmic fraction only (FIG. 9C and FIG. 10). GAPDH, F1-ΔTPase, and Lamin A/C were used as cytoplasmic, mitochondrial, and nuclear markers respectively, and they were used to ensure that there was no contamination between the different fractions. Next, the nuclear localization of PHB1 was compared in control and OA chondrocytes by indirect immunofluorescence against PHB1. Results showed a stronger signal for PHB1 in nuclei from OA chondrocytes compared to nuclei from control chondrocytes (FIGS. 9D-E). It is clear from FIG. 10 that the more severely affected subjects OA224 et OA241 (KL4) and subject OA49 (KL3) show the most significant PHB1 nuclear accumulation. Subject OA237 (KL3) also shows a higher PHB1 nuclear accumulation than OA240 (KL2) and ctrl.

Since PHB1 features both MTS (mitochondrial targeting sequence) and NES (nuclear export signal) domains, it was investigated whether PHB1 nuclear accumulation in OA articular chondrocytes was due to mutations affecting those domains by direct sequencing and no mutations were found (data not shown).

Finally, to determine whether nuclear accumulation of PHB1 in OA chondrocytes was due to an increased PHB1 expression, quantitative real-time RT-PCR analysis was performed to quantify PHB1 expression, as well as Western blot analysis of whole cell extracts. No variation in PHB1 expression levels between control and OA groups was detected (FIGS. 9F-G). Taken together, these results showed that PHB1 accumulates specifically in nuclei of OA articular chondrocytes, and this, without any change in PHB1 expression.

### Example 8

### PHB1 Level is Higher in Knee Joint Articular Chondrocytes Nuclei of Aging STR-ORT Mice

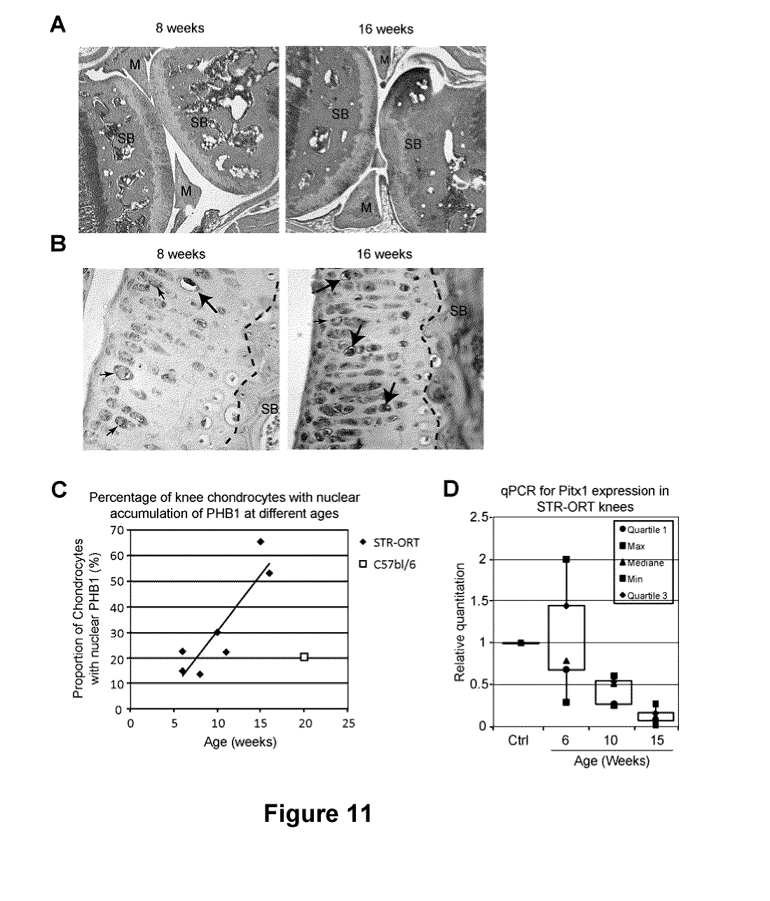

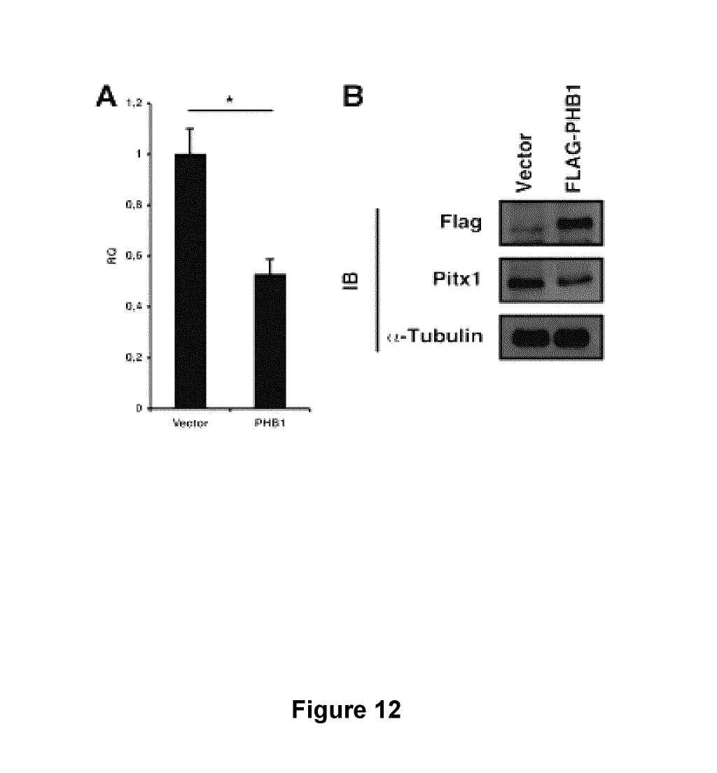

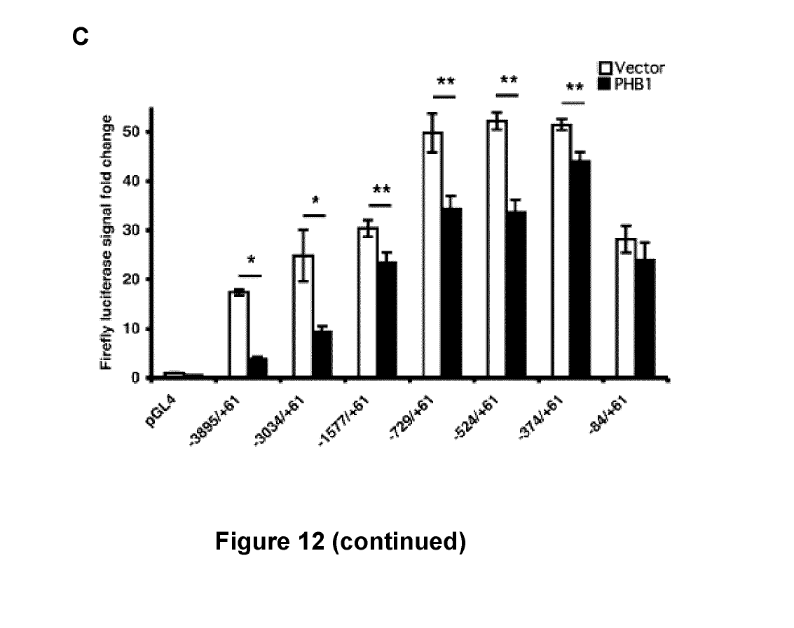

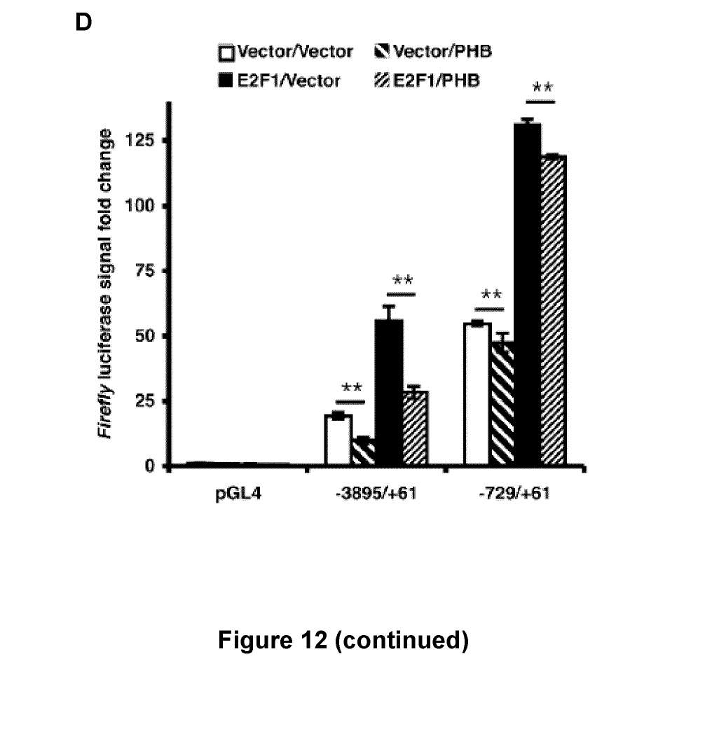

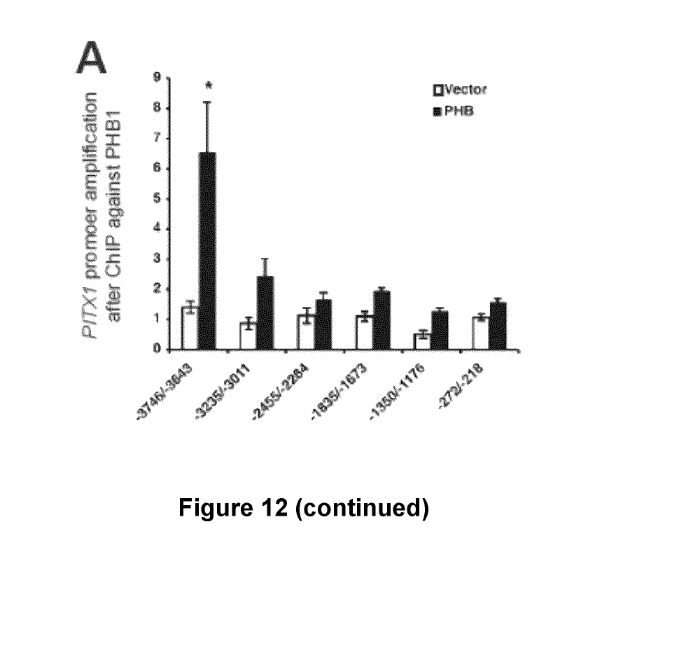

The results presented in FIG. 11 show that there is an increase in nuclear accumulation of PHB1 in knee joint articular chondrocytes of aging STR-ORT mice, a mouse model of osteoarthritis. Osteoarthritis symptoms in STR-ORT mice are known to occur at about week 30. Between weeks 8 and 16, osteoarthritis symptoms have not yet occurred (e.g., loss of proteoglycans in joints has not yet occurred, FIG. 11A) while PHB1 level has already increased (FIGS. 12B and C). These experiments show that chondrocytes nuclear PHB1 level in the mouse model starts increasing before osteoarthritis symptoms appear.

### Example 9

### PHB1 Represses Pitx1 Expression Essentially by Acting at the Distal Region of the Promoter

FIG. 12C depicts results demonstrating that over-expression of PHB1 in co-transfection assays with a Pitx1-promoter-luciferase construct −3895/+61 harboring a distal E2F-like site showed a strong repression of luciferase reporter gene (fragments −3895/+61 and smaller Pitx1 promoter fragments −3034/+61, −1577/+61, −729/+61, −524/+61 and −374/+61), thus showing that PHB1 represses Pitx1 expression essentially by acting at the distal region of the promoter. FIG. 12D shows that PHB1 blocks the effect of E2F1 with the Pitx1-promoter-luciferase reporter construct −3895/+61. These results strongly suggest that the binding of Pitx1 repressor proteins to the E2F site of the Pitx1 promoter inhibits Pitx1 expression, which in turn leads to the development/progression of osteoarthritis.

### Example 10

### Rescue of Pitx1 Expression in OA Articular Chondrocytes Through PHB1 Inhibition

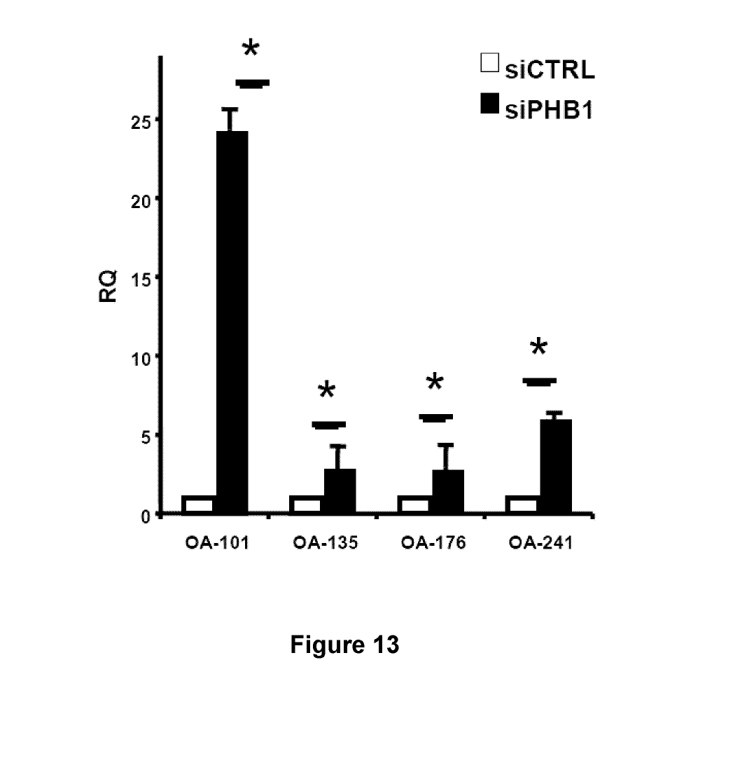

Treatment of chondrocytes from OA patients with a PHB1 siRNA, but not with a control siRNA, leads to a rescue of Pitx1 expression (FIG. 13). Knock down of PHB1 indicated a 3 to 25 fold increase in PITX1 expression in articular chondrocytes derived from OA patients.

### Example 11

### SUMO Proteins Accumulate in Nuclei of OA Articular Chondrocytes

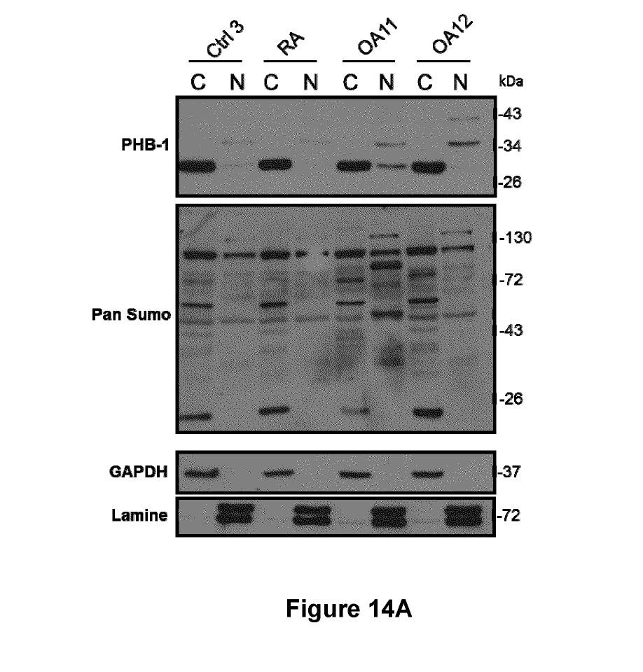

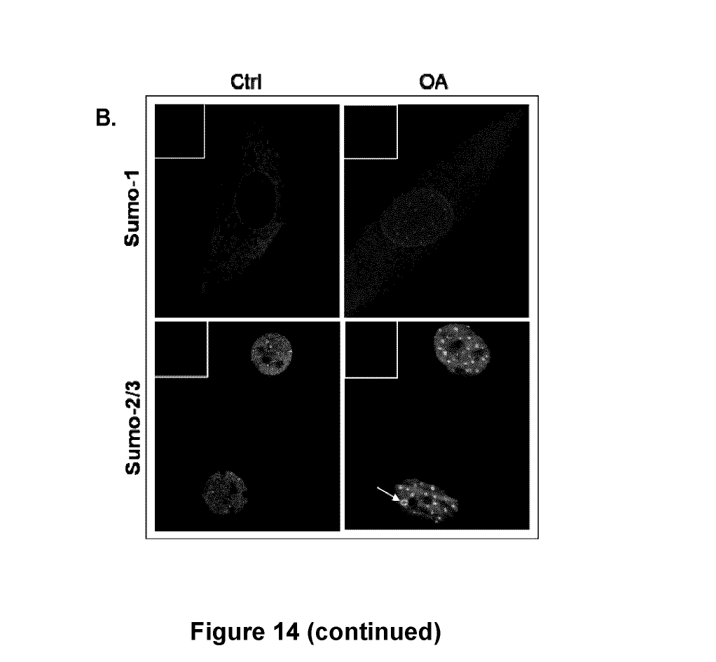

FIG. 14A compares PHB1 and Pan Sumo expression in cytoplasmic extract (C) and nuclear extract (N) of articular chondrocytes of a non arthrosic subject (Ctrl 3), a rheumatoid arthritis subject (RA) and 2 OA patients. FIG. 14B shows that OA chondrocytes display a strong nuclear accumulation of SUMO-1 and SUMO-2/3 proteins in the nuclear bodies.

### Example 12

### SUMO-1 Protein Accumulates and Co-Localizes with PHB1 in the Nuclei of Articular Chondrocytes of OA Patients

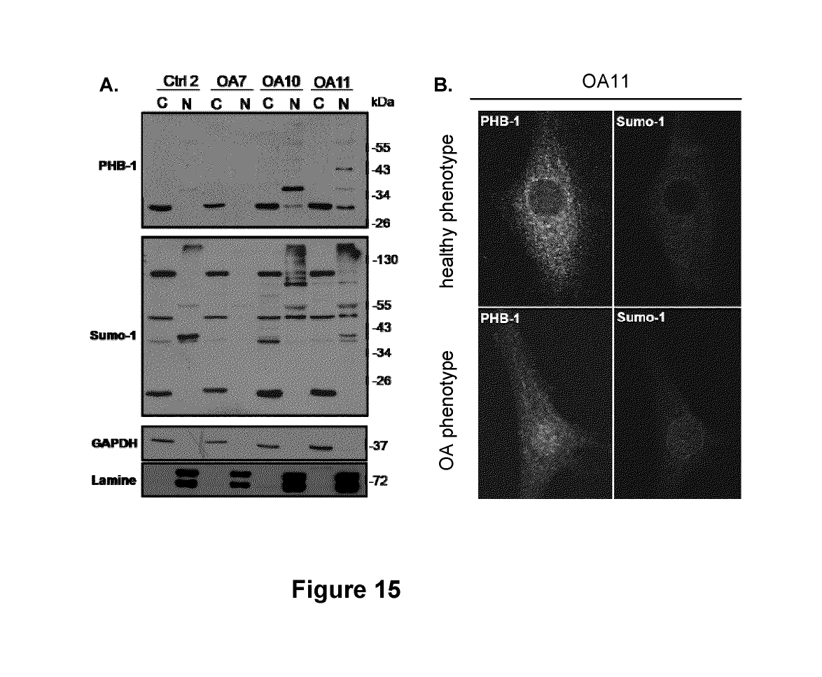

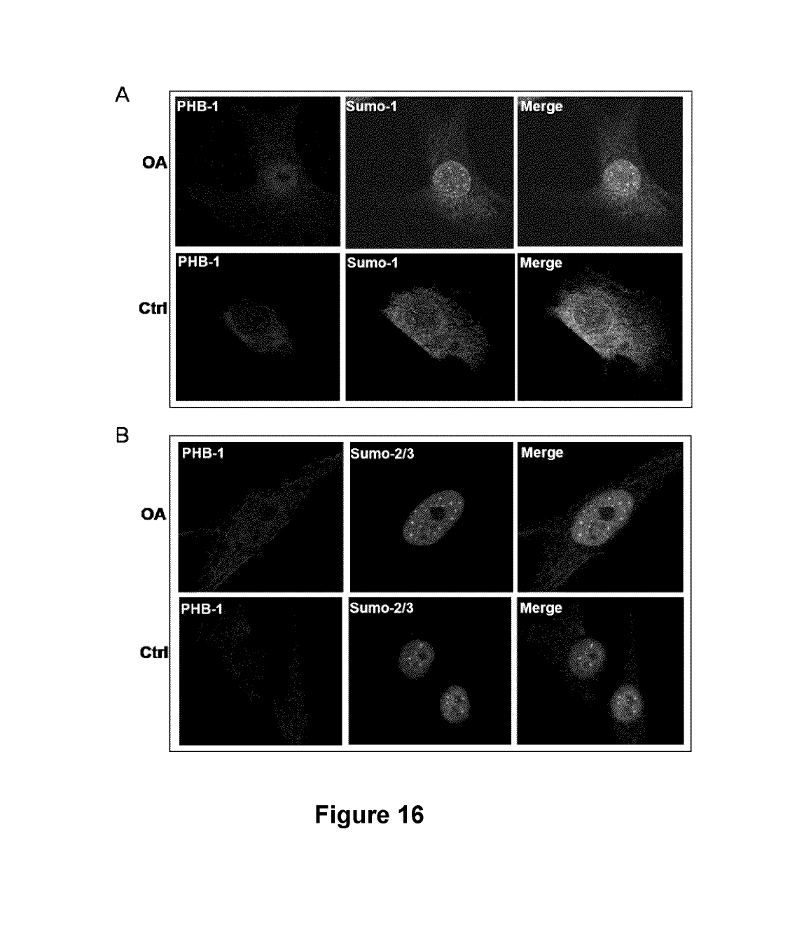

Immunoblot analysis using nuclear and cytosolic extracts purified from human articular chondrocytes of OA patients and control subjects against PHB1 and SUMO-1 revealed a band around 43 kDa (MW of PHB1 is 32 kDa and SUMO-1 is 11.6 kDa) which is consistent with the molecular weight observed with anti-PHB1 antibody at the same position, suggesting that PHB1 was sumoylated in OA11 (FIGS. 16A and 16B). Sumoylated proteins are generally difficult to detect by Western blot because the reagents and steps for sample preparation alter the sumoylation and reduce their detection. Nevertheless, the 43 kDa band is visible in the OA11 patient which may be more severely affected than the two other patients. Immunofluorescence (IF) experiments also revealed an increased accumulation of SUMO-1 and SUMO-2/3 proteins in nuclei of articular chondrocytes of OA subject OA11 (FIGS. 15B, 16A and 16B).

### Example 13

### SUMO Proteins Accumulate in PML Nuclear Bodies in OA Articular Chondrocytes

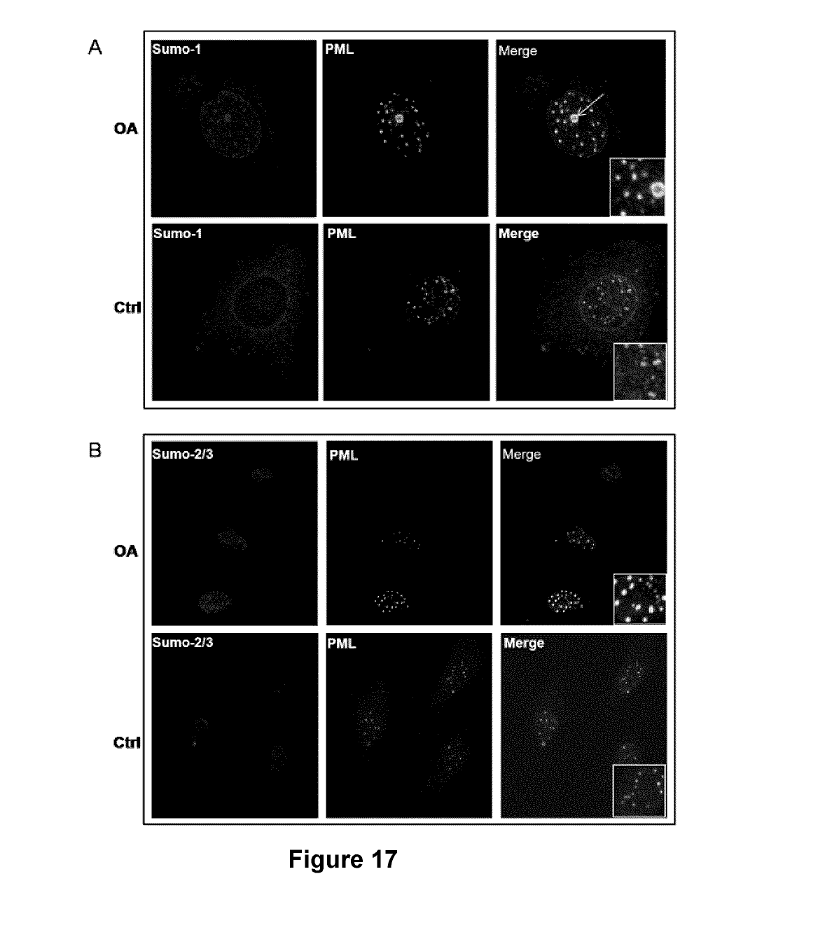

To study the intracellular localization of PHB1, SUMO-1, SUMO 2/3 and PML nuclear bodies in OA patients, IF staining combined with confocal microscopy of human articular chondrocytes in OA patients and control subjects were performed with corresponding antibodies. Double fluorescence staining with antibodies anti-PHB1 and anti-SUMO-1, indicates that both proteins were co-localized in nuclei of human articular chondrocytes in OA patients whereas in control subject, PHB1 and SUMO-1 is mostly located in the cytosol (FIG. 16A). Double fluorescence staining of OA and control human articular chondrocytes with antibodies against SUMO-2/3 and PHB1 did not show a nuclear co-localization despite the fact that both proteins are accumulated in the nuclei (FIG. 16B). In contrast, double fluorescence staining with antibodies anti-PML and SUMO-1 and SUMO-2/3 indicate that all these proteins were co-localized in nuclei of human articular chondrocytes of OA patients (FIG. 17). Furthermore, in OA chondrocytes, PML nuclear bodies are different in size and sometimes they adopt a ring structure, as indicated by an arrow (FIG. 17A, upper right panel).

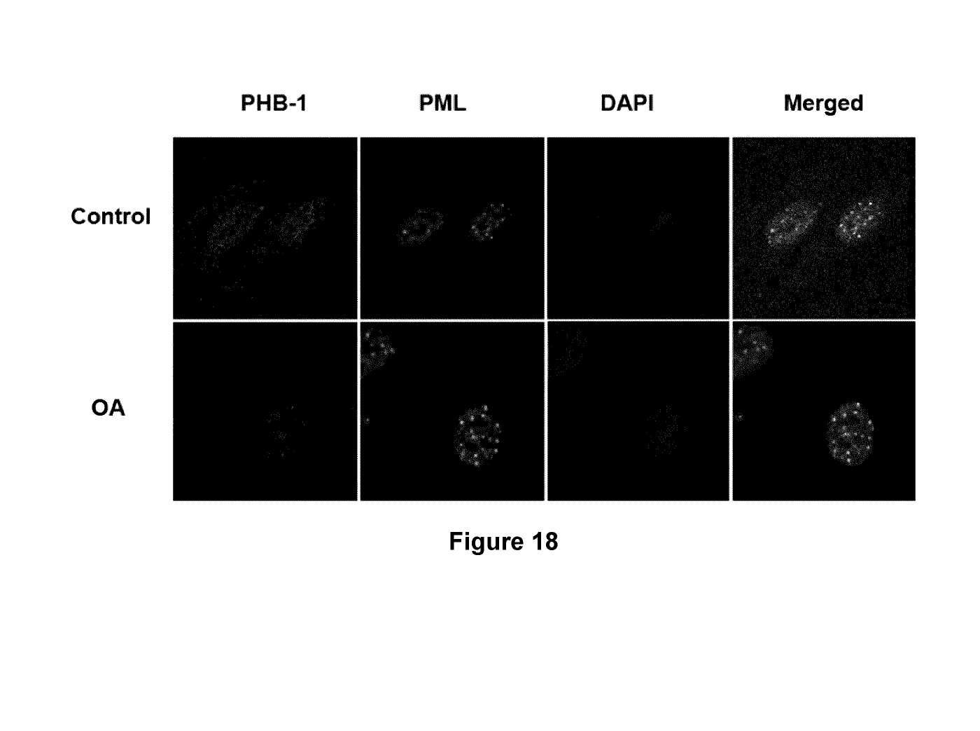

Similar IF staining experiments showed that PHB1 does not colocalize with PML nuclear bodies (See FIG. 18).

### Example 14

### SUMO1 Immunofluorescence in Mice Leucocytes

An assay was performed to determine whether STR-ort mice which develop OA with age (as do human) show an increase in SUMO1 in their leucocytes. Leucocytes were isolated from peripheral blood of 22 weeks old male mice (C57BI/6 or STR-ort) by ficoll gradient. Blood was obtained by intracardiac ponction and collected in EDTA tubes and kept at RT for less than 1 hr. before leucocytes isolation. Following their isolation on Ficoll gradiant, cells were washed in RPMI containing antibiotics and anti-mycotics but no serum. Cells were kept in this medium for about 20-30 min. (time to collect cells from all ficoll gradients+centrifugation+calculating cell concentration) before being plated on poly-D-lysine coated glass 8-well chamber slides and centrifuged at 300 g for 6 minutes, rinsed (2 quick washes) in PBS and fixed in 4% PFA for 7 minutes at room temperature. Cells were immunostained for SUMO-1 (or UBC9). Nuclei were counterstained with Draq5. Images were captured using a confocal microscope. Field of view were selected based on the Draq5 signal, focal plane was adjusted to the center of nuclei and then SUMO-1 signal was captured. Manual cell count was done using ImageJ™ cell count tool.

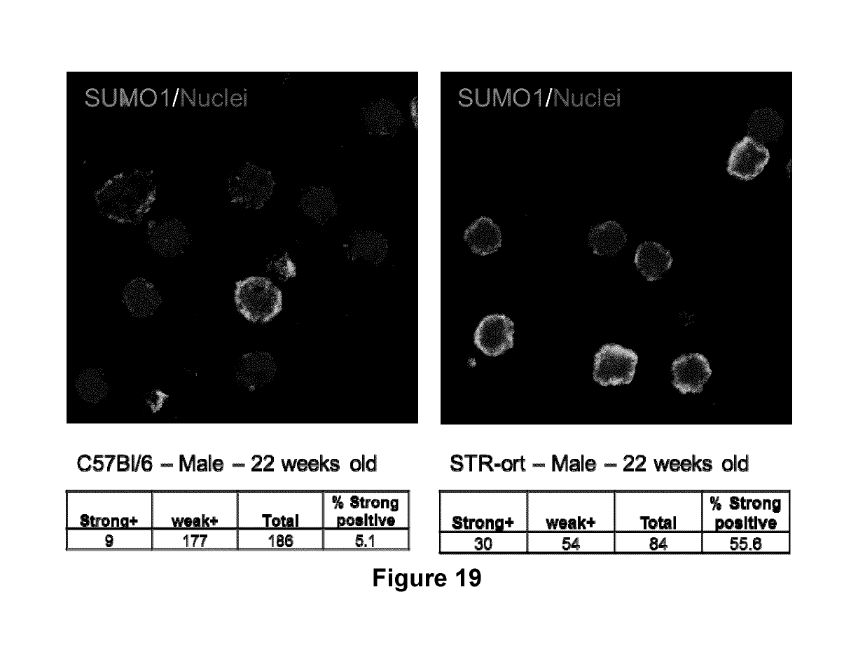

As shown in FIG. 19, the proportion of cells expressing strong positive signal for SUMO1 in 22 weeks old STR-ort male is 10× that of 22 weeks old C57BI/6 male.

Similar experiments were conducted with 7 weeks old male mice. All cells have at least some SUMO-1 signal, but the number of cells displaying clear SUMO-1 signal (that is mainly<<cytoplasmic/membrane>>) is 1 out of 31 cells in C57BI/6 mice and 9 out of 33 cells in STR/ort mice (Data not shown).

### Example 15

### Assessment of PHB1/SUMO-1 Interaction and PHB1 Sumoylation by SUMO-1 In Vitro

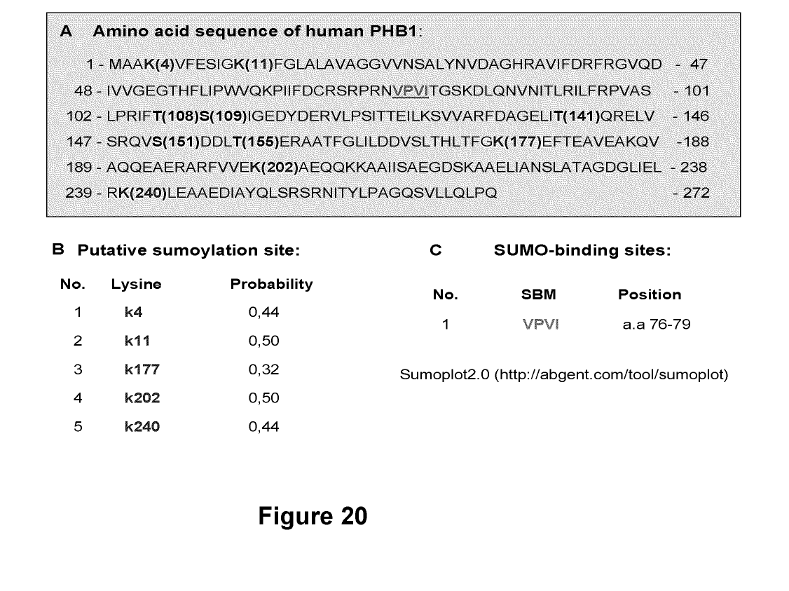

Putative sumoylation and SUMO-binding sites in human PHB1 are depicted in FIG. 20.

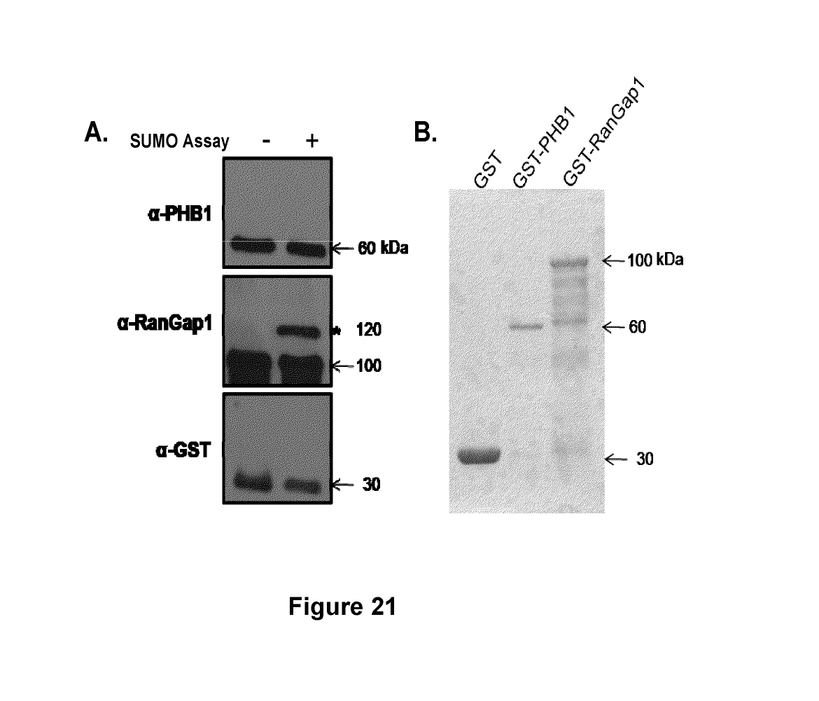

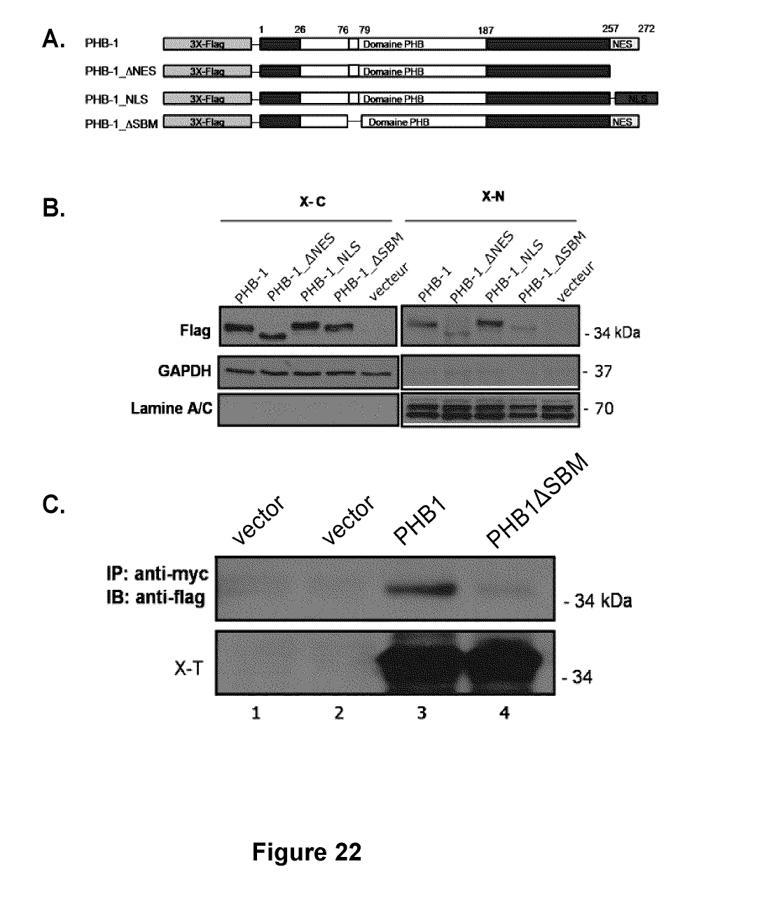

To determine whether PHB1 is directly sumoylated, a classical in vitro sumoylation assay using RanGap1 as a positive sumoylation control was performed. No evidence that PHB1 is sumoylated directly was found using this assay (FIG. 21), although PHB1 is able to bind to SUMO-1 proteins in stably-transfected C28/I2 cells (FIG. 22). Deletion of the SUMO-binding module (SBM, residues 76-79) of PHB1 significantly reduced its nuclear accumulation in transfected cells.

### Example 16

### Increased UBC9 Expression in Human Knee Joint OA Cartilage and Correlation with Disease Progression

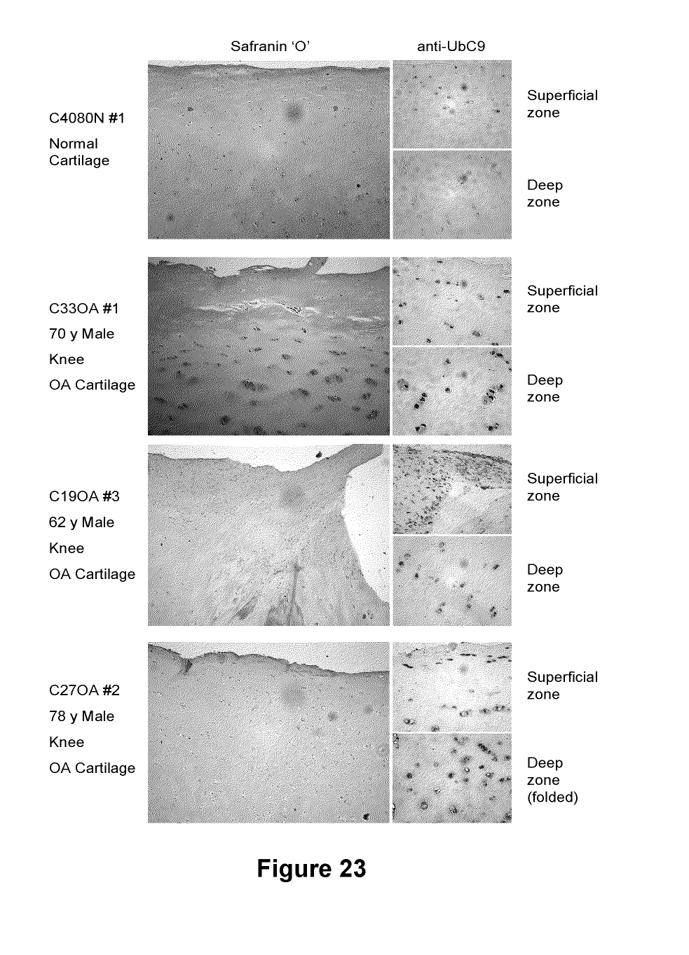

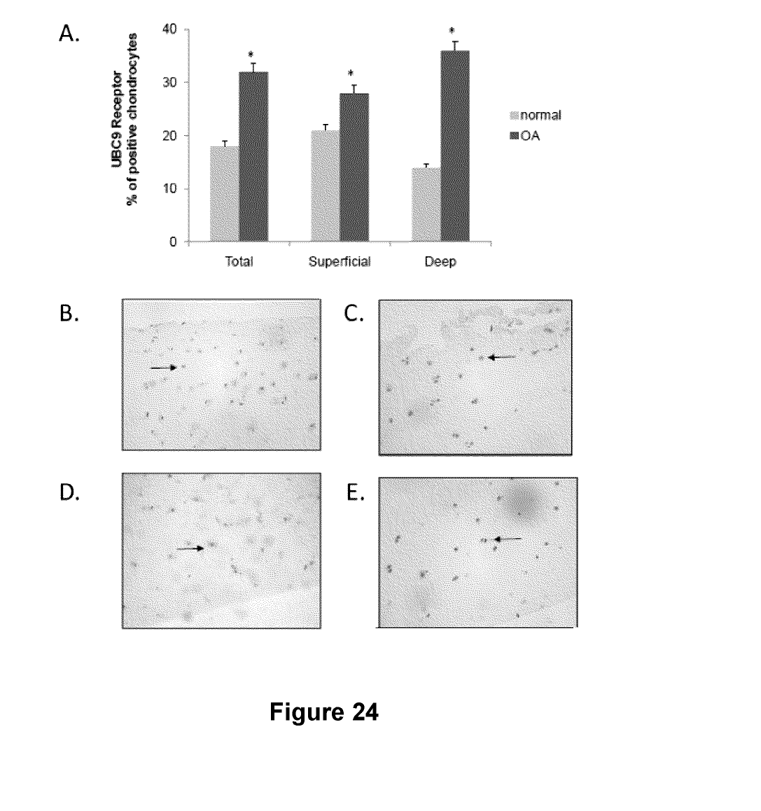

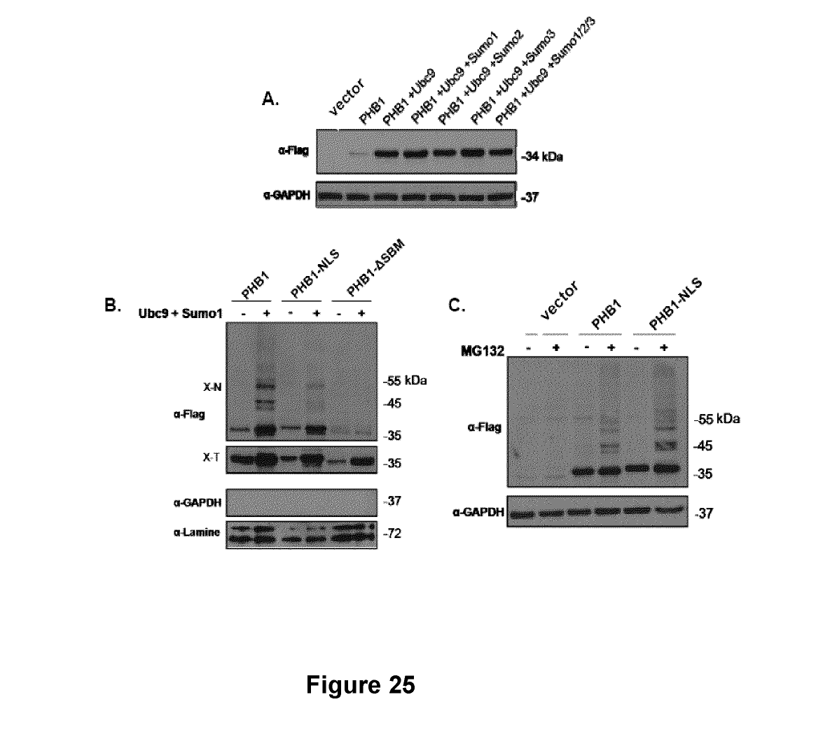

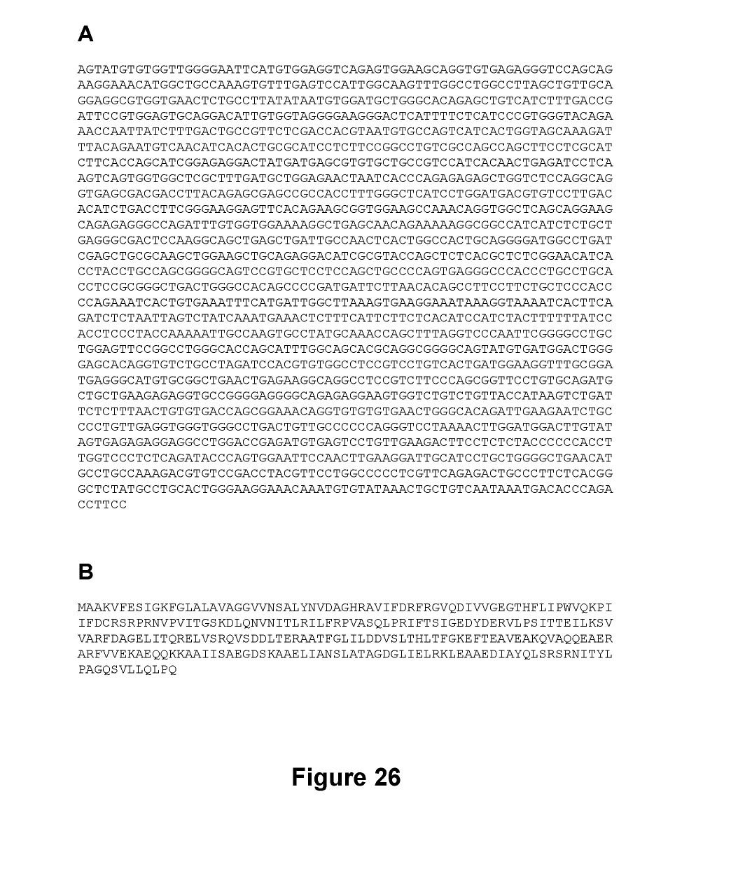

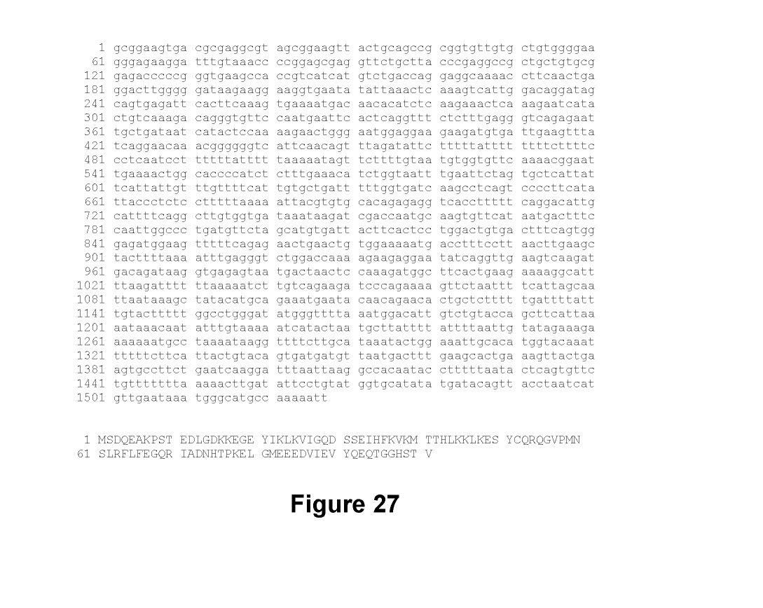

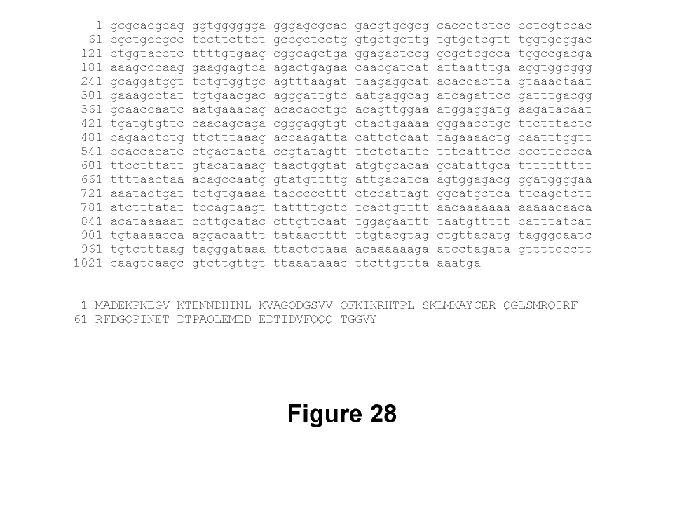

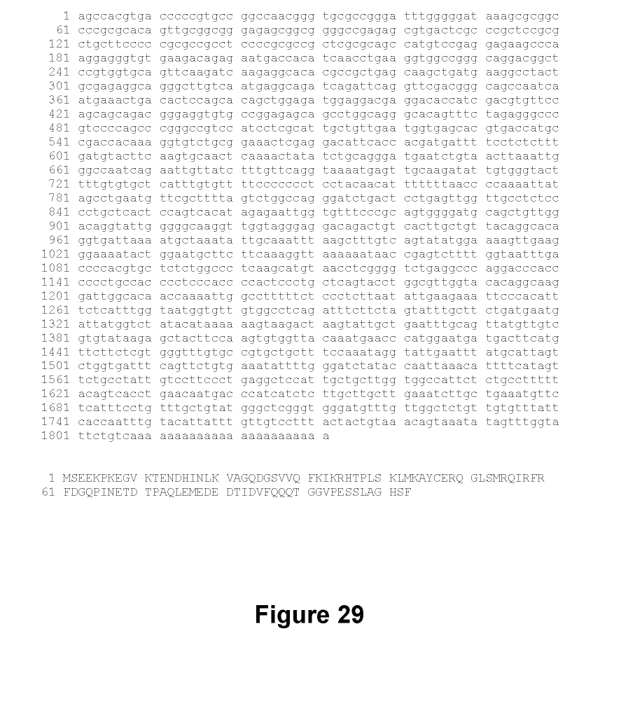

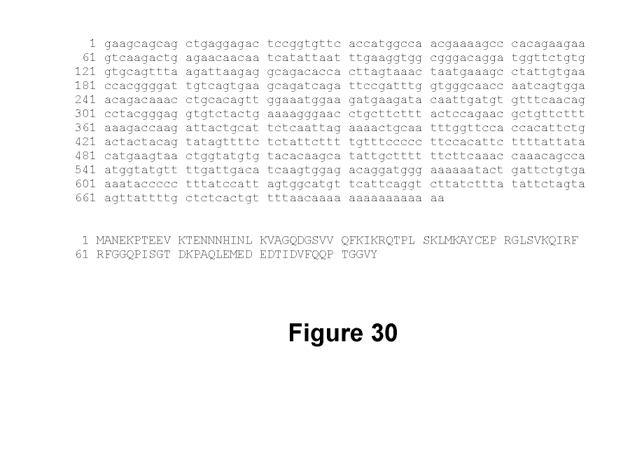

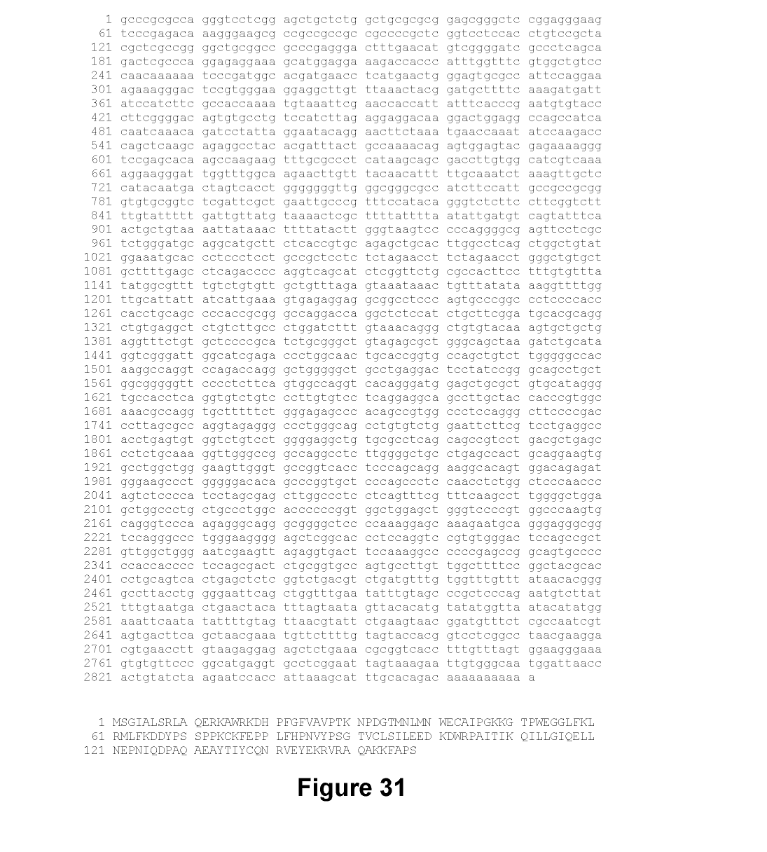

The contribution of UBC9, the E2 ligase involved in the sumoylation pathway, was investigated. The level of UBC9 protein was increased in knee joint of OA patients when compared to matched non-OA controls (FIG. 23 and FIG. 24) and correlates with disease severity as evidenced by the intensity of UBC9 IHC labelling (FIG. 25, see also FIG. 23). Overexpression of UBC9 showed that it stabilises PHB1 and promotes its nuclear accumulation in transfected U2OS cells (FIG. 25). However, this mechanism is probably indirect, as the SBM of PHB1 is involved, as evidenced by the fact that its removal in the mutant PHB1_ΔSBM abrogated the effect of UBC9 (FIG. 25B).

Although the present invention has been described hereinabove by way of specific embodiments thereof, it can be modified, without departing from the spirit and nature of the subject invention as defined in the appended claims.

### REFERENCES

1. Loughlin, J. Genetic epidemiology of primary osteoarthritis. Curr. Opin. Rheumatol. 13, 111-116 (2001).
2. Spector, T. D. & MacGregor, A. J. Risk factors for osteoarthritis: genetics. Osteoarthritis. Cartilage. 12 Suppl A, S39-S44 (2004).
3. Peach, C. A., Carr, A. J., & Loughlin, J. Recent advances in the genetic investigation of osteoarthritis. Trends Mol. Med. 11, 186-191 (2005).
4. Reginato, A. M. & Olsen, B. R. The role of structural genes in the pathogenesis of osteoarthritic disorders. Arthritis Res. 4, 337-345 (2002).
5. Loughlin, J. Polymorphism in signal transduction is a major route through which osteoarthritis susceptibility is acting. Curr. Opin. Rheumatol. 17, 629-633 (2005).
6. Drouin, J., Lanctôt, C., & Tremblay, J. J. La famille Ptx des facteurs de transcription à homéodomaine. Médecine/Sciences 14, 335-339 (1998).
7. Drouin, J., Lamolet, B., Lamonerie, T., Lanctot, C., & Tremblay, J. J. The PTX family of homeodomain transcription factors during pituitary developments. Mol. Cell Endocrinol. 140, 31-36 (1998).
8. Lanctôt, C., Lamolet, B., & Drouin, J. The bicoid-related homeoprotein Ptx1 defines the most anterior domain of the embryo and differentiates posterior from anterior lateral mesoderm. Development 124, 2807-2817 (1997).
9. Lanctôt, C., Moreau, A., Chamberland, M., Tremblay, M. L., & Drouin, J. Hindlimb patterning and mandible development require the Ptx1 gene. Development 126, 1805-1810 (1999).
10. Messeguer, X. et al. PROMO: detection of known transcription regulatory elements using species-tailored searches. Bioinformatics. 18, 333-334 (2002).
11. Farre, D. et al. Identification of patterns in biological sequences at the ALGGEN server: PROMO and MALGEN. Nucleic Acids Res. 31, 3651-3653 (2003).
12. Tommasi, S. & Pfeifer, G. P. Constitutive protection of E2F recognition sequences in the human thymidine kinase promoter during cell cycle progression. J. Biol. Chem. 272, 30483-30490 (1997).
13. Muller, H. et al. E2Fs regulate the expression of genes involved in differentiation, development, proliferation, and apoptosis. Genes Dev. 15, 267-285 (2001).
14. Zheng, N., Fraenkel, E., Pabo, C. O., & Pavletich, N. P. Structural basis of DNA recognition by the heterodimeric cell cycle transcription factor E2F-DP. Genes Dev. 13, 666-674 (1999).
15. Takahashi, Y., Rayman, J. B., & Dynlacht, B. D. Analysis of promoter binding by the E2F and pRB families in vivo: distinct E2F proteins mediate activation and repression. Genes Dev. 14, 804-816 (2000).
16. Bremner, R. et al. Direct transcriptional repression by pRB and its reversal by specific cyclins. Mol. Cell Biol. 15, 3256-3265 (1995).
17. Hamel, P. A., Gill, R. M., Phillips, R. A., & Gallie, B. L. Transcriptional repression of the E2-containing promoters EIIaE, c-myc, and RB1 by the product of the RB1 gene. Mol. Cell Biol. 12, 3431-3438 (1992).
18. Lam, E. W. & Watson, R. J. An E2F-binding site mediates cell-cycle regulated repression of mouse B-myb transcription. EMBO J 12, 2705-2713 (1993).
19. McClung, J. K., Jupe, E. R., Liu, X. T., & Dell'Orco, R. T. Prohibitin: potential role in senescence, development, and tumor suppression. Exp. Gerontol. 30, 99-124 (1995).
20. Dell'Orco, R. T., McClung, J. K., Jupe, E. R., & Liu, X. T. Prohibitin and the senescent phenotype. Exp. Gerontol. 31, 245-252 (1996).
21. Rastogi, S. et al. Prohibitin facilitates cellular senescence by recruiting specific corepressors to inhibit E2F target genes. Mol. Cell Biol. 26, 4161-4171 (2006).
22. Campisi, J. Cellular senescence as a tumor-suppressor mechanism. Trends Cell Biol. 11, S27-S31 (2001).
23. Martin, J. A. & Buckwalter, J. A. The role of chondrocyte senescence in the pathogenesis of osteoarthritis and in limiting cartilage repair. J Bone Joint Surg Am 85-A Suppl 2, 106-110 (2003).
24. Wang, S., Fusaro, G., Padmanabhan, J., & Chellappan, S. P. Prohibitin co-localizes with Rb in the nucleus and recruits N-CoR and HDAC1 for transcriptional repression. Oncogene 21, 8388-8396 (2002).
25. Altman, R. et al. Development of criteria for the classification and reporting of osteoarthritis. Classification of osteoarthritis of the knee. Diagnostic and Therapeutic Criteria Committee of the American Rheumatism Association. Arthritis Rheum. 29, 1039-1049 (1986).
26. Roy-Beaudry, M. et al. Endothelin 1 promotes osteoarthritic cartilage degradation via matrix metalloprotease 1 and matrix metalloprotease 13 induction. Arthritis Rheum. 48, 2855-2864 (2003).
27. Mankin, H. J., Dorfman, H., Lippiello, L., & Zarins, A. Biochemical and metabolic abnormalities in articular cartilage from osteo-arthritic human hips. II. Correlation of morphology with biochemical and metabolic data. J. Bone Joint Surg. Am. 53, 523-537 (1971).
28. Moreau, A., Yotov, W. V., Glorieux, F. H., & St Arnaud, R. Bone-specific expression of the alpha chain of the nascent polypeptide-associated complex, a coactivator potentiating c-Jun-mediated transcription. Mol. Cell Biol. 18, 1312-1321 (1998).
29. Deeks, J. J. & Altman, D. G. Sensitivity and specificity and their confidence intervals cannot exceed 100%. BMJ 318, 193-194 (1999).
30. Mishra S., Murphy L. C., The Prohibitins: emerging roles in diverse functions. J Cell Mol Med. 10(2): 353-63 (2006).
31. Nijtmans L G, Artal S M, Grivell L A, Coates P J. The mitochondrial PHB complex: roles in mitochondrial respiratory complex assembly, ageing and degenerative disease. Cell Mol Life Sci 2002; 59(1):143-155.
32. Mishra S, Murphy L C, Nyomba B L, Murphy L J. Prohibitin: a potential target for new therapeutics. Trends Mol Med 2005; 11(4):192-197.
33. Lawrence R C, Hochberg M C, Kelsey J L, McDuffie F C, Medsger T A Jr, Felts W R, et al. Estimates of the prevalence of selected arthritic and musculoskeletal diseases in the United States. J Rheumatol 1989; 16:427-41.
34. Felson D T, Zhang Y. An update on the epidemiology of knee and hip osteoarthritis with a view to prevention. Arthritis Rheum 1998; 41:1343-55.
35. Xavier Messeguer, Ruth Escudero, Domènec Farré, Oscar Nuñez, Javier Martínez, M. Mar Albà. PROMO: detection of known transcription regulatory elements using species-tailored searches. Bioinformatics, 18, 2, 333-334, 2002;
36. Domènec Farré, Romà Roset, Mario Huerta, José E. Adsuara, Llorenç Roselló, M. Mar Albà, Xavier Messeguer. Identification of patterns in biological sequences at the ALGGEN server: PROMO and MALGEN. Nucleic Acids Res, 31, 13, 3651-3653, 2003.
37. Gamble S C, Odontiadis M, Waxman J et al. Androgens target prohibitin to regulate proliferation of prostate cancer cells. Oncogene 2004; 23(17):2996-3004.
38. Sun L, Liu L, Yang X J, Wu Z. Akt binds prohibitin 2 and relieves its repression of MyoD and muscle differentiation. J Cell Sci 2004; 117(Pt 14):3021-3029.
39. Wang S, Zhang B, Faller D V. Prohibitin requires Brg-1 and Brm for the repression of E2F and cell growth. EMBO J 2002; 21(12):3019-3028.
40. Montano M M, Ekena K, age-Mourroux R, Chang W, Martini P, Katzenellenbogen B S. An estrogen receptor-selective coregulator that potentiates the effectiveness of antiestrogens and represses the activity of estrogens. Proc Natl Acad Sci USA 1999; 96(12):6947-6952.
41. Kurtev V, Margueron R, Kroboth K, Ogris E, Cavailles V, Seiser C. Transcriptional regulation by the repressor of estrogen receptor activity via recruitment of histone deacetylases. J Biol Chem 2004; 279(23):24834-24843.
42. Rastogi S, Joshi B, Fusaro G, Chellappan S. Camptothecin induces nuclear export of prohibitin preferentially in transformed cells through a CRM-1-dependent mechanism. J Biol Chem 2006; 281(5):2951-2959.
43. Wang S, Nath N, Fusaro G, Chellappan S. Rb and prohibitin target distinct regions of E2F1 for repression and respond to different upstream signals. Mol Cell Biol 1999; 19(11):7447-7460.
44. Wang S, Zhang B, Faller D V. BRG1/BRM and prohibitin are required for growth suppression by estrogen antagonists. EMBO J 2004; 23(11):2293-2303.
45. Wang S, Fusaro G, Padmanabhan J, Chellappan S P. Prohibitin co-localizes with Rb in the nucleus and recruits N-CoR and HDAC1 for transcriptional repression. Oncogene 2002; 21(55):8388-8396.
46. Bacher S, Achatz G, Schmitz M L, Lamers M C. Prohibitin and prohibitone are contained in high-molecular weight complexes and interact with alpha-actinin and annexin A2. Biochimie 2002; 84:1205-1218.
47. Rastogi S, Joshi B, Dasgupta P, Morris M, Wright K, Chellappan S. Prohibitin facilitates cellular senescence by recruiting specific corepressors to inhibit E2F target genes. Mol Cell Biol 2006; 26(11):4161-4171.
48. Rasmussen R K, Ji H, Eddes J S et al. Two-dimensional electrophoretic analysis of mixed lineage kinase 2 N-terminal domain binding proteins. Electrophoresis 1998; 19(5):809-817.
49. Fusaro G, Dasgupta P, Rastogi S, Joshi B, Chellappan S. Prohibitin induces the transcriptional activity of p53 and is exported from the nucleus upon apoptotic signaling. J Biol Chem 2003; 278(48):47853-47861
50. Rajalingam K, Wunder C, Brinkmann V et al. Prohibitin is required for Ras-induced Raf-MEK-ERK activation and epithelial cell migration. Nat Cell Biol 2005; 7(8):837-843.
51. Wang S, Nath N, Adlam M, Chellappan S. Prohibitin, a potential tumor suppressor, interacts with RB and regulates E2F function. Oncogene 1999; 18(23):3501-3510.
52. Lyons P., 2003. Advances in spotted microarray resources for expression profiling. Briefings in Functional Genomics and Proteomics 2, 21-30.
53. Ding G. and Cantor C. R., 2004. Quantitative analysis of nucleic acids—the last few years of progress. J Biochem Biol 37, 1-10.
54. Scheel J., Von Brevern M. C., Horlein A., Fisher A., Schneider A., Bach A. 2002. Yellow pages to the transcriptome. Pharmacogenomics 3, 791-807.
55. Lopez M. F., Pluskal M. G., 2003. Protein micro- and macroarrays: digitizing the proteome. J Chromatography B 787, 19-27.
56. Wood, W. I. et al. 1985. Base composition-independent hybridization in tetramethylammonium chloride: A method for oligonucleotide screening of highly complex gene library. Proc. Natl. Acad. Sci. USA 82. 1585-1588.
57. Kolonin et al. 2006., Nature Medicine Advance online publication of June issue.
58. U.S. Pat. No. 5,401,635.
59. Abecasis G R, Cookson W O., 2000. GOLD—graphical overview of linkage disequilibrium. Bioinformatics 16:182-3.
60. Ott, J. 2002. Predicting the range of linkage disequilibrium. Proc. Natl Acad. Sci. USA, 97, 2-3.
61. Camper-Kirby D, Welch S, Walker A et al. Myocardial Akt activation and gender: increased nuclear activity in females versus males. Circ Res 2001; 88(10):1020-1027.
62. Miller, 1988. Ann. Reports Med. Chem. 23:295.
63. Moran et al., 1987. Nucleic Acids Res., 14:5019.
64. Sambrook, J., Fritsch, E. F. & Maniatis, T. (1989). Molecular Cloning: A Laboratory Manual, Cold Spring Harbor Laboratory Press, Cold Spring Harbor, N.Y.
65. Lanctot et al., 1999. Development 126, 1805-1810.

1. A method of determining whether a subject is at risk of developing osteoarthritis (OA), said method comprising:
(A) determining the cellular localization of a Prohibitin-1 (PHB1) polypeptide and/or Small Ubiquitin-like Modifier (SUMO) polypeptide, in a cell sample from said subject; and determining whether said subject is at risk of developing OA based on the cellular localization of a PHB1 polypeptide and/or SUMO polypeptide; or
(B) determining the level of an enzyme involved in the sumoylation of protein in a cell sample from said subject; and
determining whether said subject is at risk of developing OA based on the level of said enzyme in said cell sample.

2. The method of claim 1, wherein said method further comprises:
determining whether the PHB1 polypeptide and/or SUMO polypeptide nuclear concentration is higher in the subject cell sample relative to that in a control cell sample,
wherein a higher PHB1 polypeptide and/or SUMO polypeptide nuclear concentration in the subject cell sample is indicative that the subject is at risk of developing OA.

3. The method of claim 1(A), further comprising:
determining the cellular localization of a promyelocytic leukemia (PML) polypeptide, in the cell sample from said subject,
wherein a higher level of co-localization of a SUMO-1 and/or SUMO-2 and/or SUMO-3 polypeptide and the PML polypeptide in nuclear bodies of the cell from said subject is indicative that the subject is at risk of developing OA.

4. The method of claim 1(A), wherein said SUMO polypeptide is a SUMO-1 polypeptide.

5. The method of claim 1(A), wherein said SUMO polypeptide is a SUMO-2 polypeptide.

6. The method of claim 1(A), wherein said SUMO polypeptide is a SUMO-3 polypeptide.

7. The method of claim 1(A), wherein a higher level of the SUMO polypeptide in nuclear bodies of the cell from said subject is indicative that the subject is at risk of developing OA.

8. The method of claim 7, said method comprising:
determining whether the level of co-localization of the SUMO-1 polypeptide and the PHB1 polypeptide in the nuclear bodies is higher relative to that in a control cell;
wherein a higher level of co-localization of a SUMO-1 polypeptide and a PHB1 polypeptide in nuclear bodies of the cell from said subject is indicative that the subject is at risk of developing OA.

9. (canceled)

10. The method of claim 1(B), wherein said method further comprises determining whether the level of said enzyme is higher in the subject sample relative to that in a control cell sample,
wherein a higher level of said enzyme in the subject cell sample is indicative that the subject is at risk of developing OA.

11. The method of claim 1(B), wherein said enzyme is ubiquitin-like protein sumo conjugating enzyme (UBC9).

12. The method of claim 1, wherein said cell sample is an articular chondrocyte sample, a growth plate chondrocyte sample, an osteoblast sample, a skeletal myoblast sample, a synoviocyte sample or a blood cell sample.

13. The method of claim 1, wherein said cell sample is a peripheral blood mononuclear cell (PBMC) sample.

14. The method of claim 1, wherein said cell sample is a leucocytes sample.

15. A method of determining whether a subject is at risk of developing osteoarthritis (OA), said method comprising:
determining the level of PHB1 in a blood sample from said subject; and
determining whether said subject is at risk of developing OA based on the level of PHB1 in said blood sample,
wherein a lower level of PHB1 in the subject blood sample is indicative that the subject is at risk of developing OA.

16. The method of claim 1, further comprising identifying a subject suspected of having OA.

17. The method of claim 1, further comprising identifying a subject suspected of having primary OA.

18. The method of claim 1, wherein the OA is knee joint arthritis, hip joint arthritis or temporo-mandibular joint arthritis.

19. The method of claim 18, wherein the OA is knee joint arthritis.

20. The method of claim 18, wherein the OA is hip joint arthritis.

21. The method of claim 1, wherein the OA is primary OA.

22. The method of claim 1, wherein the determining of whether the subject is at risk of developing OA determines whether the subject is at risk of developing a more severe primary OA symptoms at a future time.

23. The method of claim 1, wherein the subject is a woman.

24. A kit comprising a ligand specific to a Prohibitin-1 (PHB1) polypeptide and/or Small Ubiquitin-like Modifier (SUMO) polypeptide, and/or UBC9 polypeptide and instructions to use the ligand to predict whether a subject is at risk for developing osteoarthritis.

25. The kit of claim 24, comprising at least two of a ligand specific to a Prohibitin-1 (PHB1) polypeptide, a ligand specific to a Small Ubiquitin-like Modifier (SUMO) polypeptide, and a ligand specific to a UBC9 polypeptide.

26. The kit of claim 24, comprising a ligand specific to a Prohibitin-1 (PHB1) polypeptide, a ligand specific to a Small Ubiquitin-like Modifier (SUMO) polypeptide, and a ligand specific to a UBC9 polypeptide.


SEQUENCE LISTING (22 sequences)

SEQ ID NO: 1 - DNA, 1826 bp, homo sapiens
agtatgtgtggttggggaattcatgtggaggtcagagtggaagcaggtgtgagagggtccagcagaaggaaacatggctgccaaagtgtttgagtccattggcaagtttggcctggccttagctgttgcaggaggcgtggtgaactctgccttatataatgtggatgctgggcacagagctgtcatctttgaccgattccgtggagtgcaggacattgtggtaggggaagggactcattttctcatcccgtgggtacagaaaccaattatctttgactgccgttctcgaccacgtaatgtgccagtcatcactggtagcaaagatttacagaatgtcaacatcacactgcgcatcctcttccggcctgtcgccagccagcttcctcgcatcttcaccagcatcggagaggactatgatgagcgtgtgctgccgtccatcacaactgagatcctcaagtcagtggtggctcgctttgatgctggagaactaatcacccagagagagctggtctccaggcaggtgagcgacgaccttacagagcgagccgccacctttgggctcatcctggatgacgtgtccttgacacatctgaccttcgggaaggagttcacagaagcggtggaagccaaacaggtggctcagcaggaagcagagagggccagatttgtggtggaaaaggctgagcaacagaaaaaggcggccatcatctctgctgagggcgactccaaggcagctgagctgattgccaactcactggccactgcaggggatggcctgatcgagctgcgcaagctggaagctgcagaggacatcgcgtaccagctctcacgctctcggaacatcacctacctgccagcggggcagtccgtgctcctccagctgccccagtgagggcccaccctgcctgcacctccgcgggctgactgggccacagccccgatgattcttaacacagccttccttctgctcccaccccagaaatcactgtgaaatttcatgattggcttaaagtgaaggaaataaaggtaaaatcacttcagatctctaattagtctatcaaatgaaactctttcattcttctcacatccatctacttttttatccacctccctaccaaaaattgccaagtgcctatgcaaaccagctttaggtcccaattcggggcctgctggagttccggcctgggcaccagcatttggcagcacgcaggcggggcagtatgtgatggactggggagcacaggtgtctgcctagatccacgtgtggcctccgtcctgtcactgatggaaggtttgcggatgagggcatgtgcggctgaactgagaaggcaggcctccgtcttcccagcggttcctgtgcagatgctgctgaagagaggtgccggggaggggcagagaggaagtggtctgtctgttaccataagtctgattctctttaactgtgtgaccagcggaaacaggtgtgtgtgaactgggcacagattgaagaatctgcccctgttgaggtgggtgggcctgactgttgccccccagggtcctaaaacttggatggacttgtatagtgagagaggaggcctggaccgagatgtgagtcctgttgaagacttcctctctaccccccaccttggtccctctcagatacccagtggaattccaacttgaaggattgcatcctgctggggctgaacatgcctgccaaagacgtgtccgacctacgttcctggccccctcgttcagagactgcccttctcacgggctctatgcctgcactgggaaggaaacaaatgtgtataaactgctgtcaataaatgacacccagaccttcc

SEQ ID NO: 2 - protein, 272 aa, homo sapiens
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

SEQ ID NO: 3 - DNA, 1527 bp, homo sapiens
gcggaagtgacgcgaggcgtagcggaagttactgcagccgcggtgttgtgctgtggggaagggagaaggatttgtaaaccccggagcgaggttctgcttacccgaggccgctgctgtgcggagacccccgggtgaagccaccgtcatcatgtctgaccaggaggcaaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggtcattcaacagtttagatattctttttattttttttcttttccctcaatccttttttatttttaaaaatagttcttttgtaatgtggtgttcaaaacggaattgaaaactggcaccccatctctttgaaacatctggtaatttgaattctagtgctcattattcattattgtttgttttcattgtgctgatttttggtgatcaagcctcagtccccttcatattaccctctcctttttaaaaattacgtgtgcacagagaggtcacctttttcaggacattgcattttcaggcttgtggtgataaataagatcgaccaatgcaagtgttcataatgactttccaattggccctgatgttctagcatgtgattacttcactcctggactgtgactttcagtgggagatggaagtttttcagagaactgaactgtggaaaaatgacctttccttaacttgaagctacttttaaaatttgagggtctggaccaaaagaagaggaatatcaggttgaagtcaagatgacagataaggtgagagtaatgactaactccaaagatggcttcactgaagaaaaggcattttaagattttttaaaaatcttgtcagaagatcccagaaaagttctaattttcattagcaattaataaagctatacatgcagaaatgaatacaacagaacactgctctttttgattttatttgtactttttggcctgggatatgggttttaaatggacattgtctgtaccagcttcattaaaataaacaatatttgtaaaaatcatactaatgcttattttattttaattgtatagaaagaaaaaaatgcctaaaataaggttttcttgcataaatactggaaattgcacatggtacaaattttttcttcattactgtacagtgatgatgttaatgactttgaagcactgaaagttactgaagtgccttctgaatcaaggatttaattaaggccacaatacctttttaatactcagtgttctgtttttttaaaaacttgatattcctgtatggtgcatatatgatacagttacctaatcatgttgaataaatgggcatgccaaaaatt

SEQ ID NO: 4 - protein, 101 aa, homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 5 - DNA, 1066 bp, homo sapiens
gcgcacgcagggtggggggagggagcgcacgacgtgcgcgcaccctctcccctcgtccaccgctgccgcctccttcttctgccgctcctggtgctgcttgtgtgctcgtttggtgcggacctggtacctcttttgtgaagcggcagctgaggagactccggcgctcgccatggccgacgaaaagcccaaggaaggagtcaagactgagaacaacgatcatattaatttgaaggtggcggggcaggatggttctgtggtgcagtttaagattaagaggcatacaccacttagtaaactaatgaaagcctattgtgaacgacagggattgtcaatgaggcagatcagattccgatttgacgggcaaccaatcaatgaaacagacacacctgcacagttggaaatggaggatgaagatacaattgatgtgttccaacagcagacgggaggtgtctactgaaaagggaacctgcttctttactccagaactctgttctttaaagaccaagattacattctcaattagaaaactgcaatttggttccaccacatcctgactactaccgtatagttttctctattctttcatttcccccttccccattcctttattgtacataaagtaactggtatatgtgcacaagcatattgcattttttttttttttaactaaacagccaatggtatgttttgattgacatcaagtggagacgggatggggaaaaatactgattctgtgaaaataccccctttctccattagtggcatgctcattcagctcttatctttatattccagtaagttattttgctctcactgttttaacaaaaaaaaaaaacaacaacataaaaatccttgcataccttgttcaattggagaattttaatgtttttcatttatcattgtaaaaccaaggacaattttataacttttttgtacgtagctgttacatgtagggcaatctgtctttaagtagggataaattactctaaaacaaaaaagaatcctagatagttttcccttcaagtcaagcgtcttgttgtttaaataaacttcttgtttaaaatga

SEQ ID NO: 6 - protein, 95 aa, homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 7 - DNA, 1831 bp, homo sapiens
agccacgtgacccccgtgccggccaacgggtgcgccgggatttgggggataaagcgcggccccgcgcacagttgcggcgggagagcggcggggccgagagcgtgactcgcccgctccgcgctgcttcccccgcgccgcctccccgcgccgctcgcgcagccatgtccgaggagaagcccaaggagggtgtgaagacagagaatgaccacatcaacctgaaggtggccgggcaggacggctccgtggtgcagttcaagatcaagaggcacacgccgctgagcaagctgatgaaggcctactgcgagaggcagggcttgtcaatgaggcagatcagattcaggttcgacgggcagccaatcaatgaaactgacactccagcacagctggagatggaggacgaggacaccatcgacgtgttccagcagcagacgggaggtgtgccggagagcagcctggcagggcacagtttctagagggcccgtccccagcccgggccgtccatcctcgcattgctgttgaatggtgagcacgtgaccatgccgaccacaaaggtgtctgcggaaactcgaggacattcaccacgatgattttcctctctttgatgtacttcaagtgcaactcaaaactatatctgcagggatgaatctgtaacttaaattgggccaatcagaattgttatctttgttcaggtaaaatgagttgcaagatattgtgggtacttttgtgtgctcatttgtgttttccccccctcctacaacatttttttaaccccaaaattatagcctgaatgttcgcttttagtctggccagggatctgactcctgagttggttgcctctcccctgctcactccagtcacatagagaattggtgtttcccgcagtggggatgcagctgttggacaggtattgggggcaaggttggtagggaggacagactgtcacttgctgttacaggcacaggtgattaaaatgctaaatattgcaaatttaagctttgtcagtatatggaaaagttgaagggaaaatactggaatgcttcttcaaaggttaaaaaataaccgagtcttttggtaatttgaccccacgtgctctctggccctcaagcatgtaacctcggggtctgaggcccaggacccacccccctgccacccctcccaccccactccctgctcagtacctggcgttggtacacaggcaaggattggcacaaccaaaattggcctttttctccctcttaatattgaagaaattcccacatttctcatttggtaatggtgttgtggcctcagatttcttctagtatttgcttctgatgaatgattatggtctatacataaaaaagtaagactaagtattgctgaatttgcagttatgttgtcgtgtataagagctacttccaagtgtggttacaaatgaacccatggaatgatgacttcatgttcttctcgtgggtttgtgccgtgctgctttccaaataggtattgaatttatgcattagtctggtgatttcagttctgtgaaatattttgggatctataccaattaaacattttcatagttctgcctattgtccttccctgaggctccattgctgcttggtggccattctctgcctttttacagtcacctgaacaatgacccatcatctcttgcttgcttgaaatcttgctgaaatgttctcatttcctgtttgctgtatgggctcgggtgggatgtttgttggctctgttgtgtttattcaccaatttgtacattatttgttgtcctttactactgtaaacagtaaatatagtttggtattctgtcaaaaaaaaaaaaaaaaaaaaaaaa

SEQ ID NO: 8 - protein, 103 aa, homo sapiens
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 9 - DNA, 702 bp, homo sapiens
gaagcagcagctgaggagactccggtgttcaccatggccaacgaaaagcccacagaagaagtcaagactgagaacaacaatcatattaatttgaaggtggcgggacaggatggttctgtggtgcagtttaagattaagaggcagacaccacttagtaaactaatgaaagcctattgtgaaccacggggattgtcagtgaagcagatcagattccgatttggtgggcaaccaatcagtggaacagacaaacctgcacagttggaaatggaagatgaagatacaattgatgtgtttcaacagcctacgggaggtgtctactgaaaagggaacctgcttctttactccagaacgctgttctttaaagaccaagattactgcattctcaattagaaaactgcaatttggttccaccacattctgactactacagtatagttttctctattcttttgtttcccccttccacattcttttattatacatgaagtaactggtatgtgtacacaagcatattgcttttttcttcaaaccaaacagccaatggtatgttttgattgacatcaagtggagacaggatgggaaaaaatactgattctgtgaaaataccccctttatccattagtggcatgttcattcaggtcttatctttatattctagtaagttattttgctctcactgttttaacaaaaaaaaaaaaaaaa

SEQ ID NO: 10 - protein, 95 aa, homo sapiens
MANEKPTEEVKTENNNHINLKVAGQDGSVVQFKIKRQTPLSKLMKAYCEPRGLSVKQIRFRFGGQPISGTDKPAQLEMEDEDTIDVFQQPTGGVY

SEQ ID NO: 11 - DNA, 2871 bp, homo sapiens
gcccgcgccagggtcctcggagctgctctggctgcgcgcggagcgggctccggagggaagtcccgagacaaagggaagcgccgccgccgccgccccgctcggtcctccacctgtccgctacgctcgccggggctgcggccgcccgagggactttgaacatgtcggggatcgccctcagcagactcgcccaggagaggaaagcatggaggaaagaccacccatttggtttcgtggctgtcccaacaaaaaatcccgatggcacgatgaacctcatgaactgggagtgcgccattccaggaaagaaagggactccgtgggaaggaggcttgtttaaactacggatgcttttcaaagatgattatccatcttcgccaccaaaatgtaaattcgaaccaccattatttcacccgaatgtgtacccttcggggacagtgtgcctgtccatcttagaggaggacaaggactggaggccagccatcacaatcaaacagatcctattaggaatacaggaacttctaaatgaaccaaatatccaagacccagctcaagcagaggcctacacgatttactgccaaaacagagtggagtacgagaaaagggtccgagcacaagccaagaagtttgcgccctcataagcagcgaccttgtggcatcgtcaaaaggaagggattggtttggcaagaacttgtttacaacatttttgcaaatctaaagttgctccatacaatgactagtcacctgggggggttgggcgggcgccatcttccattgccgccgcgggtgtgcggtctcgattcgctgaattgcccgtttccatacagggtctcttccttcggtcttttgtatttttgattgttatgtaaaactcgcttttattttaatattgatgtcagtatttcaactgctgtaaaattataaacttttatacttgggtaagtcccccaggggcgagttcctcgctctgggatgcaggcatgcttctcaccgtgcagagctgcacttggcctcagctggctgtatggaaatgcaccctccctcctgccgctcctctctagaaccttctagaacctgggctgtgctgcttttgagcctcagaccccaggtcagcatctcggttctgcgccacttcctttgtgtttatatggcgttttgtctgtgttgctgtttagagtaaataaactgtttatataaaggttttggttgcattattatcattgaaagtgagaggaggcggcctcccagtgcccggccctccccacccacctgcagccccaccgcgggccaggaccaggctctccatctgcttcggatgcacgcaggctgtgaggctctgtcttgccctggatctttgtaaacagggctgtgtacaaagtgctgctgaggtttctgtgctccccgcatctgcgggctgtagagcgctgggcagctaagatctgcataggtcgggattggcatcgagaccctggcaactgcaccggtgccagctgtcttgggggccacaaggccaggtccagaccagggctgggggctgcctgaggactcctatccgggcagcctgctggcgggggttcccctcttcagtggccaggtcacagggatggagctgcgctgtgcatagggtgccacctcaggtgtctgtcccttgtgtcctcaggaggcagccttgctaccacccgtggcaaacgccaggtgctttttctgggagagcccacagccgtggccctccagggcttccccgacccttagcgccaggtagagggccctgggcagcctgtgtctggaattcttcgtcctgaggccacctgagtgtggtctgtcctggggaggctgtgcgcctcagcagccgtcctgacgctgagccctctgcaaaggttgggccggccaggcctcttggggctgcctgagccactgcaggaagtggcctggctgggaagttgggtgccggtcacctcccagcaggaaggcacagtggacagagatgggaagccctgggggacacagcccggtgctcccagccctccaacctctggctcccaacccagtctccccatcctagcgagcttggccctcctcagtttcgtttcaagccttggggctggagctggccctgctgccctggcaccccccggtggctggagctgggtccccgtggcccaagtgcagggtcccaagagggcagggcggggctccccaaaggagcaaagaatgcagggagggcggtccagggccctgggaaggggagctcggcaccctccaggtccgtgtgggactccagccgctgttggctgggaatcgaagttagaggtgacttccaaaggccccccgagccggcagtgccccccaccacccctccagcgactctgcggtgccagtgccttgttggcttttccggctacgcaccctgcagtcactgagctctcggtctgacgtctgatgtttgtggtttgtttataacacggggccttacctggggaattcagctggtttgaatatttgtagcccgctcccagaatgtcttattttgtaatgactgaactacatttagtaatagttacacatgtatatggttaatacatatggaaattcaatatattttgtagttaacgtattctgaagtaacggatgtttctcgccaatcgtagtgacttcagctaacgaaatgttcttttgtagtaccacggtcctcggcctaacgaaggacgtgaaccttgtaagaggagagctctgaaacgcggtcacctttgtttagtggaagggaaagtgtgttcccggcatgaggtgcctcggaattagtaaagaattgtgggcaatggattaaccactgtatctaagaatccaccattaaagcatttgcacagacaaaaaaaaaaa

SEQ ID NO: 12 - protein, 158 aa, homo sapiens [sequence omitted]

SEQ ID NO: 13 - DNA, 36 bp, artificial sequence
  synthetic construct
gcgaattctgctgccaaagtgtttgagtccattggc

SEQ ID NO: 14 - DNA, 27 bp, artificial sequence
  synthetic construct
gcctcgagtcactggggcagctgagga

SEQ ID NO: 15 - DNA, 36 bp, artificial sequence
  synthetic construct
gcctcgagtcagcccactttgcgctttttcttgggg

SEQ ID NO: 16 - DNA, 23 bp, artificial sequence
  synthetic construct
ttccgagagcgtgagagctggta

SEQ ID NO: 17 - DNA, 35 bp, artificial sequence
  synthetic construct
gcctcgagtcagttccgagagcgtgagagctggta

SEQ ID NO: 18 - DNA, 36 bp, artificial sequence
  synthetic construct
ccacgtaatactggtagcaaagatttacagaatgtc

SEQ ID NO: 19 - DNA, 34 bp, artificial sequence
  synthetic construct
gctaccagtattacgtggtcgagaacggcagtca

SEQ ID NO: 20 - DNA, 31 bp, artificial sequence
  synthetic construct
acggatccccgcctcggaagacattgccaag

SEQ ID NO: 21 - DNA, 32 bp, artificial sequence
  synthetic construct
atgaattcgaccttgtacagcgtctgcagcag

SEQ ID NO: 22 - protein, 271 aa, homo sapiens
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ